(19) United States
(12) Patent Application Publication
(10) Pub. No.: US 2023/0190917 A1
(43) Pub. Date: Jun. 22, 2023

(54) VIRAL VACCINE VECTOR FOR IMMUNIZATION AGAINST A BETACORONAVIRUS

(71) Applicants: Alexandru Adrian Hennrich, Munchen (DE); Karl-Klaus Conzelmann, Grafelfing (DE)

(72) Inventors: Alexandru Adrian Hennrich, Munchen (DE); Karl-Klaus Conzelmann, Grafelfing (DE)

(21) Appl. No.: 17/925,988

(22) PCT Filed: May 19, 2021
(86) PCT No.: PCT/EP2021/063273

§ 371 Date: Nov. 17, 2022

(30) Foreign Application Priority Data
May 20, 2020 (EP) 20175797.8

(51) Int. Cl. A61K 39/215; A61P 31/14; C12N 15/86

(52) U.S. Cl. CPC A61K 39/215; A61P 31/14; C12N 15/86; A61K 2039/5258

(57) ABSTRACT

The present invention relates to a composition comprising (a) a recombinant rhabdovirus vector capable of forming a virus particle and expressing an immunogen of a betacoronavirus, wherein the immunogen comprises at the C-terminus a heterologous transmembrane anchor for the incorporation of the immunogen into (i) the cell membrane of infected cells, and (ii) the envelope of the virus particle, and/or (b) a glycoprotein (G) protein gene deleted and in trans G protein complemented recombinant rhabdovirus vector capable of forming a virus-like particle (VLP) and expressing an immunogen of a betacoronavirus, wherein the immunogen comprises at the C-terminus a heterologous transmembrane anchor for the incorporation of the immunogen into (i) the cell membrane of infected cells, and (ii) the VLP.

The present invention relates to a composition comprising (a) a recombinant rhabdovirus vector capable of forming a virus particle and expressing an immunogen of a betacoronavirus, wherein the immunogen comprises at the C-terminus a heterologous transmembrane anchor for the incorporation of the immunogen into (i) the cell membrane of infected cells, and (ii) the envelope of the virus particle, and/or (b) a glycoprotein (G) protein gene deleted and in trans G protein complemented recombinant rhabdovirus vector capable of forming a virus-like particle (VLP) and expressing an immunogen of a betacoronavirus, wherein the immunogen comprises at the C-terminus a heterologous transmembrane anchor for the incorporation of the immunogen into (i) the cell membrane of infected cells, and (ii) the VLP.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Vaccines can comprise inactivated or live attenuated versions of the disease-causing viruses, heterologous virus vectors expressing immunogenic proteins of the target virus, subunit vaccines comprising virus-like particles (VLP), or individual immunogenic proteins, or DNAs or mRNAs encoding immunogenic proteins of the virus.

As is known from previous coronavirus research, inactivated and live attenuated coronavirus vaccine preparations are poorly effective, and, more importantly, pose the risk of antibody dependent enhancement of disease (ADE), when it comes to a natural infection of vaccinated persons. DNA preparations or synthetic stabilized mRNAs may be readily available but suffer from poor delivery and insufficient expression of antigens, and may require boost immunizations. Protein subunit vaccines usually require multiple sequential immunizations (boosts), which is often not practicable in reality.

Currently, there are no vaccines available for the immunization against a coronavirus, in particular against the novel beta coronavirus SARS-CoV-2 (severe acute respiratory syndrome coronavirus 2). The problems to be solved in making an efficient SARS-CoV-2 vaccine are numerous, including the choice of the best delivery system (e.g. virus vector versus VLP?), of the best antigen and its presentation (full spike (S) protein, receptor-binding domain (RBD) of S, membrane bound or soluble protein, or rather E or N proteins,) and reducing the size of the antigen to allow effective expression levels without losing important epitopes and antigenicity, and to minimize antibody-dependent enhancement (ADE) of disease

Currently more than 272 SARS-CoV-2 vaccine candidates are in development (https://www.who.int/publications/m/item/draft-landscape-of-covid-19-candidate-vaccines). Meanwhile several COVID-19 vaccines are approved. Front-runner Covid-19 vaccines employ obviously innocuous mRNA delivery for expression of a prefusion-stabilized form of the S antigen (Corbett et al., 2020; Polack et al., 2020) or replication incompetent adenoviruses (Sadoff et al., 2021). Auspiciously, these combinations turned out to be safe, and hold great promise in combating the pandemics. Many proposed COVID-19 vaccine candidates, however, employ unmodified S protein, existing in pre- and post-fusion forms and/or are based on replication competent viruses, including VSV. While it fortunately turns out that some approved SARS-CoV-2 vaccines can stimulate immune responses protecting from COVID-19 disease without overt immediate side effects and fundamentally contribute to future containment of the pandemic (see e.g. (Corbett et al., 2020; Dagan et al., 2021; Polack et al., 2020; Sadoff et al., 2021)), numerous and diverse vaccine candidates are being developed to meet the need for rapid protection of humans of all ages and conditions and/or preventing virus transmission. Prudent and transparent assessment of antigens, adjuvants and delivery vehicles is critical to prevent medical hazards and to inspire public confidence in vaccines.

Human-to-human transmission of SARS-CoV-2 and COVID-19 was confirmed on Jan. 20, 2020 and since then disease developed into a worldwide pandemic. Almost all countries are affected. As of May 18, 2020 more than 4.7 million people were diagnosed with a SARS-CoV-2 infection and more than 300,000 of these people died due to the COVID-19 disease being cause by SARS-CoV-2. In April 2021, 142.118.571 global cases were reported and 3.030.557 people have died of COVID-19.

There is therefore an urgent need for the provision of a composition that can be used to immunize people against a betacoronavirus infection and to treat people having a betacoronavirus infection. This need in addressed by the present invention.

Accordingly, the present invention relates in first aspect to a composition comprising (a) a recombinant rhabdovirus vector capable of forming a virus particle and expressing an immunogen of a betacoronavirus, wherein the immunogen comprises at the C-terminus a heterologous transmembrane anchor for the incorporation of the immunogen into (i) the cell membrane of infected cells, and (ii) the envelope of the virus particle, and/or (b) a glycoprotein (G) protein gene deleted and in trans G protein complemented recombinant rhabdovirus vector capable of forming a virus-like particle (VLP) and expressing an immunogen of a betacoronavirus, wherein the immunogen comprises at the C-terminus a heterologous transmembrane anchor for the incorporation of the immunogen into (i) the cell membrane of infected cells, and (ii) the VLP.

The term “comprise/comprising” is generally used in the sense of include/including, that is to say permitting the presence of one or more features or components. The terms “comprise” and “comprising” also encompass the more restricted terms “consist of” and “consisting of”.

As used in the specification and claims, the singular form “a”, “an” and “the” include plural references unless the context clearly dictates otherwise.

The term “composition” as used herein refers to a composition of matters comprising at least two ingredients or constituents, wherein one ingredient or constituent is the recombinant rhabdovirus vector of the invention. The other ingredient or constituent is not particularly limited and may be, for example, an excipient, aqueous solvent or water. Suitable excipients are well-known in the art and may be, for example, found in the FDA database for Inactive Ingredient Search for Approved Drug Products (https://www.accessdata.fda.gov). Also the nature of the composition is not particularly limited. The composition may be, for example, a solution or a lyophilisate and is preferably a solution.

The rhabdoviridae include 18 genera and many unassigned species (like Moussa virus). Viruses assigned to each genus form a monophyletic clade based on phylogenetic analyses of L protein sequences and usually have similar genome organizations, including the number and locations of accessory genes. Rhabdoviruses have been isolated from a wide range of vertebrates and plants; many have been isolated from arthropods. Members of the genus Lyssavirus infect mammals, including humans in which they can cause fatal encephalitis (rabies). Members of the genera Vesiculovirus, Ephemerovirus, Tibrovirus, Hapavirus, Curiovirus, Sripuvirus and Ledantevirus infect vertebrates (mammals, birds or reptiles) and are transmitted by arthropods. Some arthropod-borne rhabdoviruses are associated with diseases of livestock; some may cause disease in humans. Members of the genus Tupavirus have only been isolated from vertebrates. Members of the genera Novirhabdovirus, Sprivivirus and Perhabdovirus infect only fish, some causing economically important diseases. Rhabdoviruses assigned to the genus Sigmavirus each infect only dipteran flies of a single species and they are transmitted vertically. Members of the genus Almendravirus replicate only in insects. Plant rhabdoviruses are assigned to the genera Cytorhabdovirus, Nucleorhabdovirus, Dichorhavirus and Varicosavirus and are transmitted by either arthropods or chytrid fungi. Many are associated with diseases of agricultural or horticultural importance. The rhabdovirus which is used for the generation of the recombinant rhabdovirus vector of the invention is a rhabdovirus which does not cause a disease in humans. For instance, rabies is caused by lyssaviruses, including the rabies virus and Australian bat lyssavirus. Such natural virulent viruses are therefore not used. Only attenuated rabies viruses or G-gene deleted viruses will be used. Attenuated rabies viruses may include but are not limited to viruses with mutations of residue Arg 333 of the G protein and/or mutations in the dynein light chain binding motif of P protein (Mebatsion, 2001).

The recombinant rhabdovirus vectors to be used in accordance with items (a) and (b) of the invention are both viral vectors that can be used to deliver genetic material into cells and this genetic material is in accordance with the invention the immunogen of a betacoronavirus. The cells can be inside a living organism (in vivo) or in cell culture (in vitro).

In accordance with item (a) the recombinant rhabdovirus vector not only encodes and expresses the immunogen but also comprises and expresses the genes encoding the viral proteins required to form a rhabdovirus virus particle, noting that a rhabdovirus virus particle is capable of infecting cells and propagating the virus infection. The viral vector in accordance with the invention, thus, comprises the genes to be transferred into a cell and the viral sequences encoding the viral proteins to recognize and package the viral genome into infectious virus particles. The entire infectious virus particle consists of the nucleic acid and an outer shell of protein (called capsid) including a lipid envelope containing glycoproteins. The rhabdovirus virus particle is therefore capable of efficiently transporting the genetic information being encoded by the recombinant rhabdovirus vector into the cells being infected by the recombinant rhabdovirus virus particle. Hence, the recombinant rhabdovirus vector to be used in accordance with the invention encodes an infectious recombinant rhabdovirus virus particle which is capable of delivering and expressing genes, or other genetic material, being encoded by the vector into a target cell. This process of infection is termed transduction and the infected cells are also described as transduced cells in the art.

The recombinant rhabdovirus vector of item (a) may comprise the entire genome of a naturally occurring rhabdovirus virus and in addition a nucleic acid sequence encoding the immunogen. The virus particle being encoded by such a vector acts as a natural rhabdovirus virus in that it is mobile genetic information in an autonomously replicating and spreading and contagious infectious entity, which can infect cells and can produce more viruses without complementation of any additional genes in trans or cis.

The recombinant rhabdovirus vector of item (a) may also comprise the entire genome of a pseudotyped rhabdovirus and in addition a nucleic acid sequence encoding the immunogen. A pseudotyped virus particle is carrying one or more foreign/heterologous surface spike/glycoprotein(s) in its envelope which can mediate infection of cells (i.e. attachment to cellular receptors and membrane fusion). Examples are VSV with the glycoprotein (G) of Ebola virus (VSV-ZEBOV vaccine) or the glycoprotein (G) of rabies virus. The heterologous glycoprotein(s) can be encoded in the genome of the virus (in cis) or provided in trans from transfected plasmids or stable cell lines.

The recombinant rhabdovirus vector of item (a) may alternatively comprise the entire genome of a mosaic rhabdovirus and in addition a nucleic acid sequence encoding the immunogen. A mosaic virus particle carries a mixture of glycoproteins in its envelope. All or only one glycoprotein may be functional to allow entry into target cells. Since the glycoprotein (G) mediates the attachment of the virus particle to the host cell it is essential for the infectivity of a virus particle. The use of more than one functional glycoprotein may increase the repertoire of cells that can be infected.

In accordance with item (b) the recombinant rhabdovirus vector not only encodes and expresses the immunogen but also comprises and expresses the genes encoding the viral proteins required to form a VLP. The recombinant rhabdovirus vector of item (b) is G protein deleted and in trans G protein complemented. The term “deleted G protein” as used herein refer to any genetic manipulation of the genetic information of the recombinant rhabdovirus vector, so that the recombinant rhabdovirus vector no longer encodes and/or expresses a functional G protein capable of mediating the infection of a target cell. As discussed, the glycoprotein is essential for the infection of cells. In place of the deleted G protein a functional G protein is to be provided in trans. The step of the provision of a functional G protein in trans usually takes place ex vivo. For instance, a G protein deleted recombinant rhabdovirus vector is to be combined with a plasmid that expresses a functional glycoprotein or a transgenic cell that expresses a functional glycoprotein. The plasmid or the transgenic cell provides the glycoprotein in trans with a functional G protein, so that ex vivo an infectious virus particle is formed (noting that “cis” is used in the art to indicate that a functional G protein is encoded by the recombinant rhabdovirus vector itself). Because the recombinant rhabdovirus vector per se is G protein deleted the vector does not constitute mobile genetic information that can spread to further cells upon the initial transduction event of the described G complemented viral particles that were produced in vitro. For this reason a vector of item (b) is also called “single round infectious recombinant rhabdovirus vector”. It can express genes encoded by the virus, but does not encode a functional glycoprotein/spike in cis, such that in vivo (i.e. where no functional G protein is present for complementation in trans) no infectious virus particles are formed or infectious novel single round viruses are released from the initially infected cell. After the first round of infection the virus cannot propagate any further. Single round viruses are, thus, completely safe in particular in terms of virus spread and dissemination in the host.

Thus, instead of infectious virus particles, the recombinant rhabdovirus vector of item (b) is capable of forming a virus-like particle (VLP), noting that further details on VLPs will be provided herein below.

Hence, in accordance with item (b) the recombinant rhabdovirus vector may comprise the genome of a naturally occurring rhabdovirus virus but not a functional glycoprotein and in addition a nucleic acid sequence encoding the immunogen.

While the step of the provision of a functional G protein in trans usually takes place ex vivo, as described above, it should be noted that functional glycoproteins can also be provided in vivo, e.g. by co-infection with an unrelated virus vector like AAV encoding the glycoprotein. This is a well-established strategy in neuroscience to allow transmission from an initially RABVΔG infected neuron to a second, presynaptic neuron, but not further. This is known in the art as monosynaptic tracing of neurons (Ghanem & Conzelmann, 2016), and could be applied to allow single cycle rhabdovirus vector vaccines to perform another round of cell infection.

A rhabdovirus being complemented in trans with a functional glycoprotein is also referred to herein as “pseudotyped” virus, e.g. VSVΔG complemented with VSV G. The virus may be designated, for example, VSVΔG(VSV G) where the term in brackets indicates the origin of the glycoprotein provided in trans. Similarly, a virus called RABVΔG (SAD G) would be decorated with the G from the SAD strain of rabies.

The term “recombinant” as used in items (a) and (b) indicates that the rhabdovirus vector does not occur in nature but has been produced by laboratory methods.

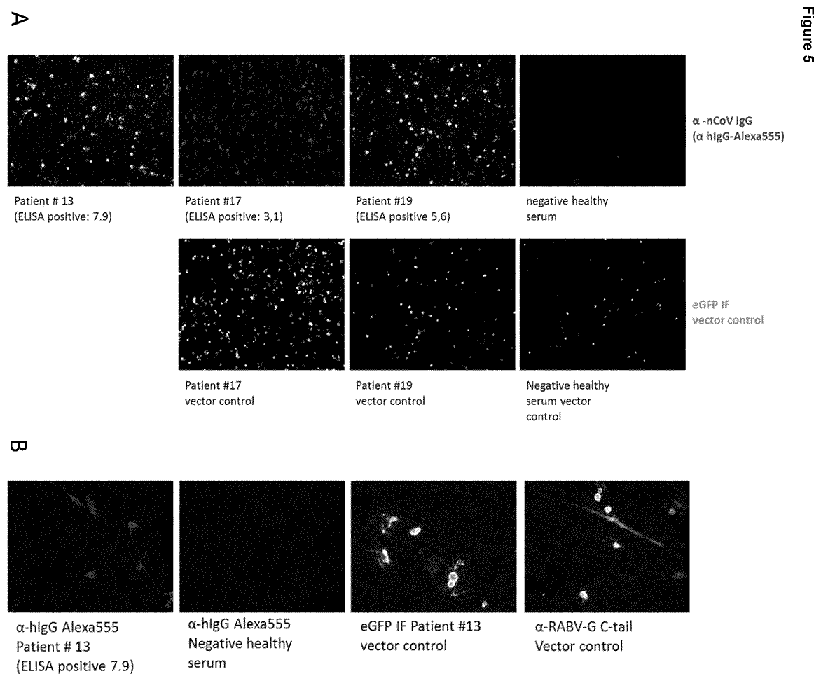

The recombinant rhabdovirus vector of items (a) and (b) expresses an immunogen of a betacoronavirus and therefore the recombinant rhabdovirus vector comprises in expressible form at least one copy of a nucleic acid molecule encoding the immunogen. The recombinant rhabdovirus vector may also comprise two, three or four or more copies of the nucleic acid molecule encoding the immunogen. The recombinant rhabdovirus vector preferably comprises one or two copies of the nucleic acid molecule encoding the immunogen as is illustrated in the appended examples (see also FIG. 4). In the case of two or more copies the two or more copies may be inserted at one site or at different sites of the recombinant rhabdovirus vector. In the first case the expression is preferably controlled via one expression control element (e.g. a rhabdovirus transcription signal sequence or promoter). By incorporating two or more copies of the nucleic acid molecule encoding the immunogen into the vector the amount of the expressed immunogen may be increased, as illustrated in FIG. 9. It is also possible to express different immunogens, at least two immunogens, e.g. from emerging SARS-CoV-2 variants, two different immunogenic fragments of the same protein of a betacoronavirus, or immunogens from different coronavirus proteins, or from two different betacoronaviruses. Further details on this aspect will be provided herein below.

A virus particle has to be held distinct from a virus-like particle (VLP). VLPs are molecules that closely resemble infectious virus particles, but are non-infectious because they contain no functional glycoprotein and usually no infectious viral genetic material. They can be naturally occurring or synthesized through the individual expression of viral structural proteins, which can then self assemble into the virus-like structure.

A VLP as used here is a rhabdovirus particle without a functional glycoprotein in its envelope. As discussed, it is released for example by cells previously transduced with a G protein complemented single round rhabdovirus vector. As the genome does not encode a functional G, but immunogens able to incorporate into the rhabdovirus envelope, as disclosed herein, non-infectious rhabdovirus particles (VLPs) will be decorated with the immunogen, and induce an immune response.

The term “expressing” means that the rhabdovirus vector comprises a nucleic acid sequence in expressible form which is expressed and subsequently translated into the immunogen within an infected cell.

Term “immunogen” refers to a molecule that is capable of eliciting an immune response by an organism's immune system. An immunogen is a particular type of an antigen. An antigen is a molecule that is capable of binding to the product of the immune system. An immunogen is therefore necessarily an antigen, but an antigen may not necessarily be an immunogen. The immunogen of a betacoronavirus is capable of eliciting an immune response by a subject's immune system against a betacoronavirus infection. The subject may suffer from a disease mediated by a betacoronavirus infection (treatment) or a disease mediated by a betacoronavirus infection may be prevented in a subject (vaccination).

Betacoronaviruses (β-CoVs or Beta-CoVs) are one of four genera of coronaviruses of the subfamily Orthocoronavirinae in the family Coronaviridae, of the order Nidovirales. They are enveloped, positive-sense, single-stranded RNA viruses of mostly zoonotic origin. The genome of a betacoronaviruses encodes four structural proteins, known as the S (spike), E (envelope), M (membrane), and N (nucleocapsid) proteins. The N protein holds the RNA genome, and the S, E, and M proteins together create the viral envelope. The spike protein is the major glycoprotein (G) of coronaviruses and as other G proteins is responsible for allowing the virus to attach to and fuse with the membrane of a host cell. The spike protein forms a crown-like structure on the surface of a coronaviruses.

The term “heterologous” as used herein in connection with a transmembrane anchor indicates that the transmembrane anchor cannot be found in a betacoronavirus and preferably in no coronavirus. Hence, an immunogen of a betacoronavirus carrying on the C-terminus a heterologous transmembrane anchor cannot be found in nature.

The term “heterologous” as used herein may also be used with respect to rhabdovirus glycoproteins. Hence, the transmembrane anchor derived from the rabies virus G protein in combination with a VSV vector or particle is heterologous.

A transmembrane anchor for the incorporation of the immunogen into (i) the cell membrane of infected cells, and (ii) the envelope of a recombinant rhabdovirus virus particle and/or a virus-like particle (VLP) is an amino acid sequence that can be incorporated into the membrane of cells as well as the membrane of virus particles of rhabdovirus (and most likely also lentiviruses and retroviruses) and VLPs derived from rhabdoviruses, lenti-, and retroviruses. A transmembrane anchor generally spans the entire membrane. Due to the presence of the transmembrane anchor at the C-terminus of the immunogen, the immunogenic is presented on the outside of an infected cell, a virus particle or a VLP upon its incorporation of the transmembrane anchor into the cell membrane. Since the transmembrane anchor of the present invention which is expressed at the cell membrane is compatible with the intracellular structure of budding rhabdoviruses like RABV and VSV, and possibly lentiviruses, the immunogen is efficiently incorporated into virions released to the extracellular space.

In more detail, due to the presence of a transmembrane anchor according to the invention at the C-terminus of the immunogen of a betacoronavirus the administration of the composition of the invention to a subject leads to the immunization of the subject and the elicitation of a immune response within the subject against a betacoronavirus infection by two fundamental different mechanisms of immunogen representation to the immune system.

On the one hand, the expressed immunogen is transported to the cell surface of cells being infected by the recombinant rhabdovirus vector according to the invention and displayed on the surface of the infected cells. This is evidenced by the data in the appended examples showing that infected cells are recognized by COVID-19 reconvalescent patient sera. Hence, the presentation of the immunogen on the cell surface is expected to induce a strong antibody response to the immunogen.

In addition to immunization by standard cell-surface expression the immunogens are also incorporated into the envelope of recombinant rhabdovirus virus particles and/or into virus-like particles (VLPs). Virus particles and are released into the extracellular space and are expected to further contribute to the overall immune stimulation against a betacoronavirus.

Of note, the envelope proteins of rhabdovirus particles like VSV are presented in a dense and strictly arranged array forming paracrystalline layers. They therefore present epitopes in a highly preferable way, namely in form of repetitive surface epitopes. It is known that repetitive antigens are highly immunogenic and can induce antibody responses even T-cell independently (Astori & Kraehenbuhl, 1996; Fehr et al., 1997; Hangartner, Zinkernagel, & Hengartner, 2006; Szomolanyi-Tsuda & Welsh, 1998). The generation of rhabdovirus particles or VLPs carrying the immunogen is therefore considered to substantially contribute to the overall success of the combined cell and virus particle immunization approach described here.

The antibodies induced by the immunogen are expected to neutralize infectivity of a betacoronavirus.

As shown herein below in the examples, vaccination of animals with a VSV replicon encoding a chimeric SARS-CoV-2 S RBD/rabies virus G transmembrane anchor results in high levels of SARS-CoV-2 neutralizing antibodies.

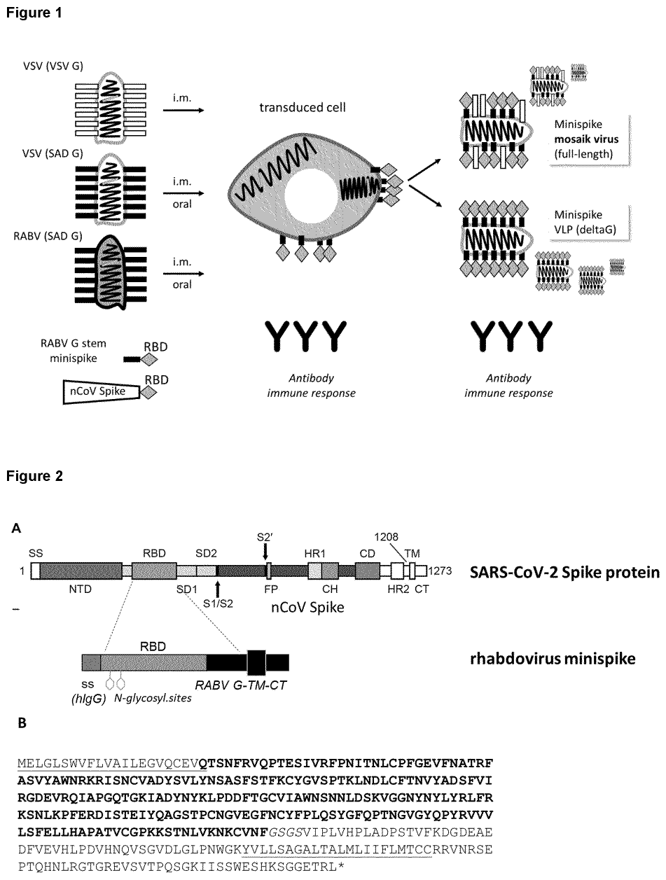

The composition of the first aspect of the invention, thus, advantageously, provides for a vaccination approach, wherein the immunogen is represented on the cell surface of infected cells, and in addition on the surface of virus particles and/or optionally VLPs (FIG. 1). These two fundamental different mechanisms of immunogen representation synergistically act together and elicit an immune response which is superior to one mechanism alone.

As also shown herein below in the examples, vaccination with the VSV-minispike replicon provides complete protection of transgenic K18-hACE2 mice from COVID-19 disease.

In accordance with a preferred embodiment of the first aspect of the invention, the betacoronavirus is selected from SARS-CoV-2, MERS-CoV, SARS-CoV-1, 0043 and HKU1, and is preferably SARS-CoV-2.

MERS-CoV causes Middle East Respiratory Syndrome (MERS), SARS-CoV-1 causes severe acute respiratory syndrome (SARS) and SARS-CoV-2 is the novel betacoronavirus that causes COVID-19. Further examples of betacoronaviruses causing respiratory infections in humans are the coronaviruses 0043 and HKU1. The five discussed betacoronaviruses are the Group 2 coronaviruses that are human pathogens. It is speculated that SARS-1, SARS-CoV-2, and MERS-CoV arose from a bat reservoir (Cui et al., 2019).

There is also immunological cross-reaction between the phylogenetically related SARS-1 and SARS-2 viruses. SARS-CoV-1 RBD-specific sera cross-reacted with SARS-CoV-2 RBD protein, and cross-neutralized SARS-CoV-2 virus. A human Mab targeting SARS-CoV-1 RBD was also found to bind, though a bit weaker, to SARS-CoV-2 RBD in ELISA, and to prevent infection of culture cells by both viruses (Tian et al., 2020) (Wang et al., 2020). The possibility of developing SARS-CoV RBD-based vaccines for simultaneous prevention of both SARS-CoV-2 and SARS-CoV infection has thus been suggested. However, some of the most potent SARS-CoV-1-specific virus neutralizing antibodies (VNAs) (e.g. m396, CR3014) failed to bind the SARS-CoV-2 spike protein (Tian et al., 2020). This indicates that the differences in the RBDs of SARS-CoVs are critical for cross-reactivity of VNAs, and that it is still desirable to develop vaccines and antibodies to specifically target at least SARS-CoV-1 and SARS-CoV-2.

In any case, it is likely that the strategy outlined here to generate an immune response is not only applicable to SARS-CoV-2 but to all betacoronaviruses. In view of be relatedness of the discussed five betacoronaviruses it is also possible that an immunogen from one of the five betacoronaviruses is also capable of eliciting an immune response against another betacoronaviruses of the five betacoronaviruses.

In accordance with another preferred embodiment of the first aspect of the invention the immunogen is the spike (S) protein or an immunogenic fragment thereof.

Antiviral compositions are mostly aimed at the production of antibodies, in particular, virus neutralizing antibodies (VNA), which can prevent entry of the virus into its target cells. Entry of enveloped viruses is mediated by viral surface proteins, which can bind to specific cellular receptors and fuse viral and cellular membranes to release the viral genetic information into the cell. As discussed, in the case of coronaviruses the spike protein (S) is mediating entry.

The pandemic SARS-CoV-2 is closely related to bat SARS-CoV RaTG13 and to a lesser degree to human SARS-CoV-1, and the proteins show an overall high homology (Wu et al., 2020). The Spike proteins of the former viruses show >97% amino acid identity (Wrapp et al., 2020) and 76% amino acid identity to SARS-1 (Hoffmann et al., 2020) and they are using the same receptor for entry, namely angiotensin converting enzyme 2 (ACE2). Of note, sera from convalescent SARS-1 patients were shown to cross-neutralize SARS-CoV-2 entry (Hoffmann et al., 2020; Tai et al., 2020), and binding of some monoclonal antibodies (Mab) made against SARS-1 S to the S protein of SARS-2 was observed (Amanat & Krammer, 2020). The more distantly related middle east respiratory syndrome virus (MERS) CoV virus spike is utilizing DPP4 (Dipeptidyl peptidase 4) as receptor, and other human beta coronaviruses can use other receptors (Cui et al., 2019; Letko, Marzi, & Munster, 2020).

While the closely related Spike proteins of SARS-1 and SARS-2 viruses have equivalent functions, a few small but important molecular changes may have led to rapid pandemic spread of SARS-CoV-2. First, a multibasic cleavage site between the S1 and S2 subunits, rather than a single Arg residue as found in SARS-1, allows faster activation of S by cellular proteases like furin and TMPRSS2 (Hoffmann et al., 2020; Wu et al., 2020). In addition, binding of the S protein to the ACE2 receptor is 10-20 fold stronger (Tai et al., 2020; Wrapp et al., 2020). This results in more efficient infection and virus production in tissue not permissive for SARS-1 virus, such as the upper respiratory tract, and enhanced human-to-human transmission (Kim et al., 2020; Rockx et al., 2020; Shi et al., 2020). Moreover, mutated variants of SARS-CoV-2 are continuously emerging, including so-called variants of interest (VOI) or variants of concern (VOC). Mutations often affect the S protein and may result in greater S protein stability, increased hACE2 binding, or even escape form pre-existing immune responses to natural infections with ancestral SARS-CoV-2 variants, or from vaccination-induced immunity (Garcia-Beltran et al., 2021; Markus Hoffmann et al., 2021) (Baum et al., 2020; Weisblum et al., 2020). As described below in the examples, vaccination with the VSV minispike replicon elicits antibodies which neutralize the most important VOCs.

In addition to the respiratory tract, SARS-CoV-2 can be detected in multiple organs, including kidneys, heart, liver, brain, and intestine (Lamers et al., 2020; Puelles et al., 2020).

As for SARS-CoV-1, the spike (S) protein of SARS-CoV-2 is the favored antigen to be comprised in any vaccine for induction of VNAs, since antibodies binding S can interfere with the correct association of S and the ACE2 receptor and the subsequent membrane fusion process (Chen, Strych, Hotez, & Bottazzi, 2020; Du et al., 2009; He et al., 2005). Indeed, S of SARS-CopV1 was found to be the only structural protein inducing significant amounts of VNAs after vaccination with a recombinant PIV3 virus (Buchholz et al., 2004).

The strategy of expressing SARS-CoV-2 Spike proteins from viral vectors, including measles virus, MVA vaccinia virus, Adenovirus, adeno-associated viruses, etc. is close at hand (Amanat & Krammer, 2020; Chen et al., 2020). In addition, VLPs carrying Spike on their surface are a viable option. Also nucleic acid based approaches based on DNA or mRNA delivery include Spike sequences. In April 2020 more than 70 candidate nCoV vaccines were in development (https://www.who.int/blueprint/priority-diseases/key-action/novel-coronavirus-landscape-ncov.pdf) (Table DRAFT landscape of COVID-19 candidate vaccines—20 Apr. 2020), (Thanh Le et al., 2020, Amanat & Krammer, 2020), and most of the candidates encompass the whole spike protein, or the S RBD. Even clinical trials have already started in the US, using stabilized mRNA encoding SARS-CoV-2 Spike for immunization of human volunteers.

For the above reasons the immunogen to be used in accordance with the present invention is preferably the spike (S) protein of a betacoronavirus or an immunogenic fragment thereof.

In view of the high similarity of S proteins from SARS-CoV-1 and SARS-CoV-2, in particular the expression of the nCoV Spike from VSV and RABV vectors is expected to result in the production of protective SARS CoV-2/COVID antibodies after immunization.

The amino acid and nucleotide sequences of the spike (S) protein of SARS CoV-2 are shown in SEQ ID NOs 2 and 3, respectively. SEQ ID NO: 4 is a human codon-optimized nucleotide sequences of the spike (S) protein of SARS CoV-2.

An obstacle to the development of effective betaCoV spike vaccines, however, is the large size of the S protein. The natural nCoV S is a huge, heavily glycosylated transmembrane protein of 1273 amino acids and >150 kDa, which may be a too heavy load for many viral vectors.

This payload may result in a suboptimal replication of the vectors and/or suboptimal expression of the immunogen from those vectors. In addition, numerous epitopes of variable antigenicity and which are located in relevant or irrelevant domains of the protein are presented to the immune system when the entire spike is used. In other words, relevant and irrelevant epitopes could compete for antibody stimulation. While the full length S may stimulate a broad variety of immune responses and antibodies, not all of those will work for inducing virus neutralizing antibodies. Some antibodies which do not neutralize virus infectivity might even be harmful and cause antibody dependent enhancement (ADE) of disease after natural infection like those induced by antigenic sites on the envelope glycoproteins of HIV and Ebola virus (Jiang, Lin, & Neurath, 1991; Nakayama et al., 2011; Takada, Watanabe, Okazaki, Kida, & Kawaoka, 2001). In fact, in the absence of neutralizing antibodies, enhanced inflammation was observed in rabbits after MERS reinfection (Houser et al., 2017), studies on a recombinant SARS-CoV-1 spike vaccine suggested induction of neutralizing and enhancing antibodies (Jaume et al., 2012), and non-human primates vaccinated with modified vaccinia Ankara virus (MVA) encoding full-length SARS-CoV spike glycoprotein and challenged with the SARS-CoV virus were reported to have lower viral loads but to suffer from acute lung injury due to antibody-dependent enhancement (ADE) (Liu et al., 2019). Thus, it is preferred to instruct the immune system to more specifically induce effective virus neutralizing antibodies.

Therefore, in accordance with a more preferred embodiment of the first aspect of the invention the immunogenic fragment consists of or comprises the spike receptor binding domain (RBD) and preferably comprises the fragment represented by SEQ ID NO: 1 or a variant of SEQ ID NO: 1 being with increasing preference at least 80%, at least 85%, at least 90% and at least 95% identical to SEQ ID NO: 1.

The sequence identities as referred to herein are preferably determined with BLAST (basic local alignment search tool) (blast.ncbi.nlm.nih.gov/Blast.cgi).

The most relevant domain for neutralization is believed to be the spike receptor binding domain (RBD), which directly binds to the ACE2 receptor. For instance, the RBD of SARS-CoV-2 is located in the S1 subunit of S, and is similar in sequence to that of SARS-CoV-1. Analysis of the receptor binding motif (RBM), a portion of the RBD that makes contact with ACE2 (F. Li, Li, Farzan, & Harrison, 2005; W. Li et al., 2003, Li et al., 2005a), revealed that most amino acid residues essential for ACE2 binding by SARS-S were conserved in SARS-CoV-2-S(Hoffmann et al., 2020; Shang et al., 2020; Tai et al., 2020; Yan et al., 2020). However, the SARS-CoV-2 RBD exhibits significantly higher binding affinity to human and bat ACE2 receptors than the SARS-CoV-1 RBD (Tai et al., 2020). It was shown that purified RBD constructs encompassing residues 331 to 524 (NITN . . . HAPATV (SEQ ID NOs 18 and 19)) of S protein containing a C-terminal Fc of human IgG1 (hFc) for purification by protein A affinity chromatography (Tai et al., 2020), or residues 319-591 (RVQP . . . CVNF (SEQ ID NOs 20 and 21)) [along with the S signal peptide (amino acids 1-14, 189 MFIF . . . TSGS (SEQ ID NOs 22 and 23)) plus a hexahisitidine tag] (Wrapp et al., 2020) can bind SARS reconvalescent sera antibodies and can be used for serological tests.

For the above reasons the use of a fragment consisting of or comprising the spike receptor binding domain (RBD) as the immunogen is particular advantageous. SEQ ID NO: 1 is the sequence comprising the SARS-CoV-2 RBD (i.e. amino acid positions 314 to 541 of the spike protein S) as used in the appended examples and is used in the context of an immuongen referred to herein as “minispike”. Further details on minispike will be provided herein below. The amino acid sequence of the SARS-CoV-2 RBD is shown in SEQ ID NO: 1.

Preferred examples of variants of SEQ ID NO: 1 (i.e. amino acid positions 314 to 541 of the spike protein S) comprise one or more, preferably one to three of the following mutations: K417N, K417T, L452R, E484K, E484Q, N501Y. More examples of variants of SEQ ID NO: 1 are SEQ ID NO: 1 with [E484K], [E484K, N501Y], [K417N, E484K], [K417T, E484K], [K417N, E484K, N501Y], [K417T, E484K, N501Y], or [L452R, E484K, N501Y] as well as [E484Q], [E484Q, N501Y], [K417N, E484Q], [K417T, E484Q], [K417N, E484Q, N501Y], [K417T, E484Q, N501Y], [L452R, E484Q, N501Y] or [L452R, T478K]. As is further discussed in Examples 9 and 11 these mutations in the SARS-CoV-2 RBD were identified in SARS-CoV-2 variants of concern (VOCs) in Great Britain (B.1.1.7; 20I/501Y.V1), South Africa (B.1.351; 20H/501Y.V2), Brazil (P1; 20J/501Y.V3), and India (B.1.617; 20A). The VOCs are generally more transmissible than the wild-type SARS-CoV-2.

In accordance with a further more preferred embodiment of the first aspect of the invention the immunogenic fragment consists of less than 300 amino acids, preferably less than 250 amino acids, and most preferably less than 230 amino acids

In accordance with a further more preferred embodiment of the first aspect of the invention the immunogenic fragment consists of 150 to 300 amino acids, preferably 200 to 250 amino acids, and most preferably 220 to 240 amino acids.

The immunogenic fragment of the spike protein as used in the appended examples consists of 228 amino acids. As discussed, the use of nucleic acid molecules encoding an immunogen which is too large may result in suboptimal virus replication and/or immunogen expression. As also discussed, a viral vector may comprise more than one copy of a nucleic acid molecule encoding the immunogen or two or more different nucleic acid molecules encoding different immunogens, including those of variants of the SARS-CoV-2.

Prerequisites for the design of such viral vectors is the design of nucleic acid molecules encoding immunogens which are small enough to allow the incorporation of two or more copies into one viral vector, and, on the other hand, and encoding immunogens which provide sufficient stimulation of the immune system for the development of virus neutralizing antibodies. Immunogenic fragments consisting of the above indicated number of amino acids are believed to meet these prerequisites.

Notably, the small size of minispikes and minispike genes as illustrated in the examples allows expressing multiple copies from a single vector, thus, enhancing the antigen dose expressed from a vector construct. As shown in the examples VSVdeltaG and full length VSV, expressing two or three copies of minispike (bimini, tri-miniconstructs) can be readily generated. The feasibility of expressing multiple repetitive gene sequences was previously also shown for a rabies virus live vaccines (Faber et al., 2007).

The size and the nature of the entire immunogenic part of the vector will be described in further detail in connection with the fourth aspect of the invention. The further details on immunogenic part as described in connection with the fourth aspect of the invention apply mutatis mutandis to the fist aspect of the invention.

The amino acid sequence of the entire exemplified immunogenic part (minispike construct) is shown in SEQ ID NO: 16 and this minispike construct is encoded by the nucleotide sequence of SEQ ID NO: 17. The immunogenic fragment preferably comprises or consists of SEQ ID NO: 1 which is comprised in SEQ ID NO: 16. Instead of SEQ ID NO: 1 the immunogenic fragment may also comprise or consist of a variant of SEQ ID NO: 1 as described herein above. Also described herein are amino acid sequences being with increasing preference at least 80%, at least 85%, at least 90% and at least 95% identical to SEQ ID NO: 16 or being encoded by nucleotide sequences being with increasing preference at least 80%, at least 85%, at least 90% and at least 95% identical to SEQ ID NO: 17.

In accordance with a further preferred embodiment of the first aspect of the invention the rhabdovirus is preferably rhabdovirus vesicular stomatitis virus (VSV) or rabies virus (RABV) or is a G protein gene deleted and in trans-complemented VSV or RABV (namely VSVΔG and RABVΔG, respectively).

VSV and the related RABV are well-studied prototype members of the Rhabdoviridae family (including 30 genera) within the Mononegavirales order (i.e. nonsegmented negative strand RNA viruses) (Walker et al., 2018) and belong to the Vesiculovirus and Lyssavirus genus, respectively. VSV can only cause disease in the group of ungulate animals, including livestock, but normally not in humans, primates, and other animals.

The viral RNA of VSV and RABV is comprised in a highly stable, helical nucleocapsid (NC; RNP) and the information encoded is expressed by sequential and polar transcription of discrete subgenomic mRNAs from the NCs (for review see (Abraham & Banerjee, 1976; Whelan, Barr, & Wertz, 2004). VSV and RABV genomes comprise only 5 genes (transcription units; TU) in the order 3′-N-P-M-G-L-5′. N (nucleoprotein), P (phosphoprotein) and L (Large polymerase) are engaged in RNA encapsidation (N), and RNA synthesis (L+P) using exclusively the NC as a template for viral RNA synthesis. M (matrixprotein) and G (glycoprotein) are involved in the assembly and membrane envelopment of the RNPs during budding at the cell membranes. Reverse genetics of RABV and VSV is well established (Conzelmann, 2013; Lawson, Stillman, Whitt, & Rose, 1995; M. J. Schnell, Mebatsion, & Conzelmann, 1994) and additional multiple transcription units (ATUs) encoding foreign genes (e.g. encoding foreign antigens or immunogens) can be introduced into the genomes. Both VSV and RABV are being widely used as live heterologous vaccine vectors, and recombinant VSV in addition is being used for oncolytic virotherapy (Bishnoi, Tiwari, Gupta, Byrareddy, & Nayak, 2018; Fathi, Dahlke, & Addo, 2019; Majid, Ezelle, Shah, & Barber, 2006; Zemp, Rajwani, & Mahoney, 2018). Because of the entirely cytoplasmic replication and the lack of a DNA stage, rhabdovirus vectors are considered safe with respect to possible integration of foreign sequences into the host genome.

A particularly favorable feature of rhabdoviruses like RABV and VSV is the possibility to delete the glycoprotein gene (so-called “delta G”, “dG”, “AG”, “G-deficient” viruses) and to complement the G protein in trans which yields expression vectors which cannot spread further from an initially infected cell, and which are thus completely safe.

Generally homologous G proteins (GP) can be replaced with other transmembrane proteins including heterologous viral surface proteins or “spike” or glycoproteins. This can be either in cis, or in trans. In cis, the foreign glycoproteins are encoded by the virus (surrogate viruses). If the foreign GP is functional as a virus entry protein, the tropism of the resulting recombinant VSV is determined by the heterologous GP; as illustrated by the recently approved VSV-ZEBOV vaccine, in which the VSV G gene is replaced by the EBOV GP gene (Fathi et al., 2019). Complementation of ΔG viruses in trans with functional viral glycoproteins yields infectious pseudotype virus particles which are able to perform 1 infection round but unable to spread further as no G protein is expressed in the infected cells, which could mediate infection of other cells (Ghanem & Conzelmann, 2016).

This “envelope switching” enables targeting of virus infection to specific cells in the case that functional attachment and fusion proteins are used (Matsuura et al., 2001; Mebatsion & Conzelmann, 1996; Mebatsion, Finke, Weiland, & Conzelmann, 1997; Wickersham, Lyon, et al., 2007).

Of note, the requirements for efficient foreign protein incorporation are different for RABV and VSV. While efficient RABV pseudotyping requires cytoplasmic tails similar to that of the RABV G (Mebatsion & Conzelmann, 1996), VSV is much more promiscuous and incorporates almost every abundantly expressed protein with a short cytoplasmic tail (M. J. Schnell et al., 1998), including the nCoV-Spike protein (M. Hoffmann et al., 2020), or the RABV G cytoplasmic tail (Moeschler, Locher, Conzelmann, Kramer, & Zimmer, 2016). One practical application/consequence of these findings is the following: When a foreign protein, like the SARS-2 spike, carries the RABV G cytoplasmic tail it is incorporated very well in both RABVΔG and VSVΔG pseudotype particles and VLPs.

Important differences exist also in the biology of RABV and VSV. While RABV is slowly replicating and causes prolonged infection, VSV is a typical hit and run virus, which means that it is a very rapidly replicating virus, expressing large quantities of RNA and protein and growing to very high titers. In addition to the powerful viral RNA synthesis machinery of VSV, a powerful host cells transcription shut down is executed by the VSV matrix (M) protein (Black & Lyles, 1992). This leads to preferential production of virus-encoded proteins, including foreign antigens. In other words, VSV is a high-level gene expression machine, and a VSV vaccine vector thus rapidly expresses high amounts of antigens and immunogens.

Among the options of the above preferred embodiment, the options VSV and a G protein gene deleted and pseudotyped VSV are therefore preferred. VSV and a G protein gene deleted and pseudotyped VSV are preferred “emergency vaccines”. Emergency vaccines are heterologous vector viruses able to rapidly express high amounts of antigenic proteins, such that only a single shot leads to an immune response, and which are innocuous to vaccinated persons.

Of note, there are also versions of VSV which do not shut down host gene expression, because of mutations introduced into the VSV M protein, such as M(M51R), MΔ51, or Mq (M. Hoffmann et al., 2010; Kopecky, Willingham, & Lyles, 2001; Publicover, Ramsburg, Robek, & Rose, 2006). While these vectors cannot prevent host cell shut down, they activate the intrinsic host antiviral immune response, including induction of type I and type III interferon (IFN) system, which is a powerful arm of the host antiviral defense and immune activation. VSV M mutants are favorable in certain settings. In addition, recombinant rhabdovirus vectors, including VSV and RABV, can be equipped with a control mechanism (SMASh-System), which allows stopping of virus replication by small drugs (Ghanem and Conzelmann, WO 2020/021090 A1). The ability to stop replication and spread of replication competent vaccine or oncolytic viruses in case of emergency or when their desired function is fulfilled is relevant in terms of biological safety.

In summary, recombinant VSV is a prime vaccine vector both with respect to efficacy and safety. A recombinant VSV encoding the Ebola virus glycoprotein (GP) instead of its own G protein (VSV-EBOV) was found to be highly effective in preventing Ebola virus infection and disease, as illustrated following the Ebola outbreaks in Africa. Notably, replication competent VSV-EBOV is now approved in the EU as an emergency vaccine against Ebola disease. This illustrates the superior excellence of VSV as a vaccine vector. VSV vectors are particularly well suited for intramuscular immunization.

In accordance with another preferred embodiment of the first aspect of the invention the immunogen of a betacoronavirus comprises at the N-terminus a signal peptide that promotes high-level translation into the endoplasmic reticulum, wherein the signal peptide is preferably derived from an immunoglobulin, preferably from IgG and most preferably from the heavy chain of IgG.

A signal peptide is present at the N-terminus of the majority of newly synthesized proteins that are destined towards the secretory pathway. These proteins include those that are inserted into cellular membranes. The core of the signal peptide contains a long stretch of hydrophobic amino acids (about 5-16 residues long) that has a tendency to form a single alpha-helix and is also referred to as the “h-region”. In addition, many signal peptides begin with a short positively charged stretch of amino acids, which may help to enforce proper topology of the polypeptide during translocation by what is known as the positive-inside rule. Because of its close location to the N-terminus it is called the “n-region”. At the end of the signal peptide there is typically a stretch of amino acids that is recognized and cleaved by signal peptidase and therefore named cleavage site.

Many examples of signal peptides that promote high-level translation into the endoplasmic reticulum are known in the art and the addition of such a signal peptide to the N-terminus of the immunogen of a betacoronavirus will facilitate the incorporation of the immunogen into the membrane of infected cells.

Non-limiting but preferred examples of signal peptides are signal peptides derived from an immunoglobulin, preferably from IgG and most preferably from the heavy chain of IgG.

Preferred examples of signal peptides derived from the heavy chain of IgG are shown in SEQ ID NOs 5 (signal peptide IgH HV 3-7 amino acid sequence) and 6 (signal peptide IgH HV 3-13 nucleotide sequence) which were used in the examples.

Examples of other suitable signal peptides are shown in SEQ ID NOs 7 to 13. The examples are signal peptides derived from immunoglobulin heavy variable 3-21 (IGHV3-21), immunoglobulin heavy variable 1-46 (IGHV1-46), high affinity immunoglobulin gamma Fc receptor I (FCGR1A), IgG receptor FcRn large subunit p51 (FCGRT), serum albumin (ALB), prolactin (PRL) and Azurocidin preproprotein (AZU1), respectively.

In accordance with an further preferred embodiment of the first aspect of the invention the transmembrane anchor is derived from the stem of a rhabodovirus glycoprotein, and preferably derived from members of the Lyssavirus genus, more preferably derived from the rabies (RABV) vaccine strain SAD B19 (molecular clone SAD L16), and most preferably comprises or consists of the membrane proximal part of the G ectodomain, the trans-membrane and the cytoplasmic tail of the RABV SAD L16 G protein.

The minispike constructs as used in the examples combine immunogenic parts of the SARS-CoV-2 S protein, in particular the RBD, and structural parts derived from the stem of a rhabdovirus G protein as carrier for the RBD antigen.

The “stem” of a rhabdovirus G protein is the part of the a rhabdovirus G protein which forms the transmembrane anchor for the incorporation of the immunogen into (i) the cell membrane of infected cells, and (ii) the envelope of a recombinant rhabdovirus virus particle and, if present, a virus-like particle (VLP).

In particular the stems of the rhabdovirus G proteins from the Lyssavirus genus have been reported to result in a trimeric form of the G protein on the virus particle surface (Buthelezi et al. (2016), Virology, 498:250-256). Moreover, also the spike proteins of coronaviruses assemble into trimers on the virus particle surface to form the discussed distinctive corona. It is therefore believed that in particular the use of the stems of a rhabdovirus G protein from the Lyssavirus genus results in the formation of an immunogen having a trimeric structure which is incorporated well in a rhabdovirus envelope, including the RABV envelope from which it stems. The trimeric structure of the minispike allows the presentation of the immunogen to closely resemble that of the naturally occurring spike protein of a betacoronavirus. This is in turn deemed advantageous in order to achieve an efficient protective immune response involving the formation of virus neutralizing antibodies.

The stem of the rhabdovirus G protein as used in the examples is derived from the rabies (RABV) vaccine strain SAD B19 (molecular clone SAD L16). This stem consists of the membrane proximal part of the G ectodomain, the trans-membrane and the cytoplasmic tail of the RABV SAD L16 G protein. The stem as used in the examples is most preferred and has the amino acid sequence of SEQ ID NO: 14 and is encoded by SEQ ID NO: 15. The stem may also have amino acid sequences being with increasing preference at least 80%, at least 85%, at least 90% and at least 95% identical to SEQ ID NO: 14.

SEQ ID NO: 14 reads
GSGSVIPLVHPLADPSTVFKDGDEAEDFVEVHLPDVHNQVSGVDLGLPN
WGKYVLLSAGALTALMLIIFLMTCCRRVNRSEPTQHNLRGTGREVSVTP
QSGKIISSWESHKSGGETRL,

noting that the double-underlined amino acids are a synthetic linker and that the the membrane spanning portion of the anchor is underlined. A schematic illustration of the minispike construct is provided in FIG. 2

Using this stem allows generation of both VSV and RABV VLPs in case of ΔG vectors, and of mosaic VSV and RABV viruses in case non-deficient virus vectors are used, as is illustrated below in the examples and figures.

In accordance with a yet further preferred embodiment of the first aspect of the invention the recombinant rhabdovirus vector comprises at least two, preferably at least three copies of the nucleotide sequence encoding the immunogen of a betacoronavirus, wherein the vector comprises these copies at one site or at different sites of the vector.

As discussed herein above, recombinant rhabdovirus vector which comprises at least two, preferably at least three copies of the nucleotide sequence encoding the immunogen of a betacoronavirus is advantageous since more copies of the immunogen per viral vector are expressed. This in turn is expected to further enhance the strength of the anti-viral immune response.

The recombinant rhabdovirus may comprise the nucleotide sequence(s) encoding the immunogens of a betacoronavirus at any gene position (#1, #2, #3, #4, #5, or #6) in the genome, either en bloc [i.e. three minispikes between G and L] or dispersed (i.e. one at pos. 2, one at pos. 4, etc.), noting that the standard rhabdogenome is (3′-N-P-M-G-L-S′) e.g. #1: 3′-s-N-P-M-G-L, #5: (3′-N-P-M-G-s-L). In case of delta G viruses, a possible position for insertion of the minispike gene is #4, i.e. the place of the deleted G gene.

The small size of the minispike construct facilitates not only the introduction of multiple minispike copies, but also the further introduction of other immunogenic parts of the spike protein and/or immunogenic parts of the coronavirus N, M, or E proteins.

In accordance with another preferred embodiment of the first aspect of the invention, the composition additionally comprises a VLP, wherein the VLP is preferably a G protein gene deleted rhabdovirus which is not complemented in trans with a functional G protein gene.

In the case of G protein deficient viral vector complementation with attachment- and/or fusion-incompetent membrane proteins or with only parts of glycoproteins, or just immunogens, virus like particles (VLPs) are generated. If the composition comprises immunogens as described here, and which are compatible for incorporation into rhabdovirus envelopes, VLPs decorated with the immunogen will be generated and released from transduced cells. Hence, such VLPs can be used to increase the concentration and abundance of extracellular immunogens in a subject upon the administration to the subject.

In accordance with a further preferred embodiment of the first aspect of the invention, the composition is a pharmaceutical composition or a diagnostic composition and is preferably a vaccine composition.

In accordance with the present invention, the term “pharmaceutical composition” relates to a composition for administration to a subject, preferably a human subject. The pharmaceutical composition of the invention comprises the compound recited above. It may, optionally, comprise further molecules capable of altering the characteristics of the compounds of the invention thereby, for example, stabilizing, modulating and/or activating its function. The composition may be in solid, liquid or gaseous form and may be, inter alia, in the form of (a) powder(s), (a) tablet(s), (a) solution(s) or (an) aerosol(s). The pharmaceutical composition of the present invention may, optionally and additionally, comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, organic solvents including DMSO etc. Compositions comprising such carriers can be formulated by well known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the ordinary clinician or physician. Generally, the regimen as a regular administration of the pharmaceutical composition should comprise the recombinant rhabdovirus vector of item (a) in amount of about 1000 to about 100000 CCID₅₀ (Cell Culture Infectious Dose 50%) and/or the rhabdovirus vector of item (b) in amount of about 10⁵ to about 10⁸ FFU (focus forming units). The term “about” is preferably +/−20% and most preferably +/−10%.

The length of treatment needed to observe changes and the interval following treatment for responses to occur vary depending on the desired effect. The particular amounts may be determined by conventional tests for B and T cell immune responses, which are well known to the person skilled in the art. A vaccine composition is a particular form of a pharmaceutical composition that provides an active and acquired immunity to a particular infectious disease to subject receiving the vaccine.

In accordance with the present invention, the term “diagnostic composition” relates to a composition for identifying the presence of a disease in a subject, preferably a human subject. Otherwise a diagnostic composition may be formulated in a similar way as a pharmaceutical composition. The difference is the other intended use of these two compositions. In the present case the subject may be checked for the presence of antibodies against the immunogen.

The present invention relates in a second aspect to the composition of the first aspect for use in preventing or treating a betacoronavirus infection and preferably a SARS-CoV-2 infection.

The definitions and preferred embodiments of the above first aspect apply mutatis mutandis to the second aspect of the invention.

Also described herein is a method for the treatment or the prevention of a betacoronavirus infection and preferably a SARS-CoV-2 infection in a patient in need thereof comprising administering to the patient a therapeutically effective amount of the composition of the invention to the subject.

As discussed herein above, the administration of the composition of the invention initiates an immune response against a betacoronavirus infection in a subject. The composition of the invention is therefore suitable for the treatment and prevention of a betacoronavirus infection and in particular for the vaccination against a betacoronavirus infection.

In accordance with a preferred embodiment of the second aspect of the invention the composition is to be administered intramuscularly.

As discussed above, the recombinant rhabdovirus vector is preferably a VSV vector. VSV vectors are particularly suited for intramuscular (i.m.) administration. For i.m. immunization, VSV is recommended and preferred, as it is a high-level expression machine in muscle cells, but RABV vectors are also suitable for i.m. vaccination.

In accordance with another preferred embodiment of the second aspect of the invention the composition is to be administered orally.

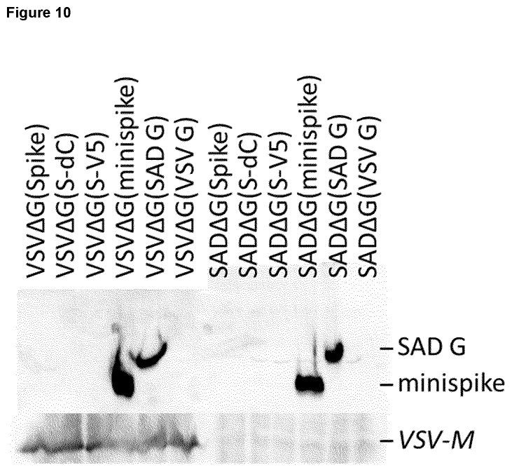

RABV strains like SAD and EU-approved SAD-derived live vaccines like Rabitec® are particularly useful for oral vaccination, in particular for oral immunization. Oral vaccination with minispike-encoding replicating rabies-based vaccines is thus feasible. A more preferred embodiment is using G-deficient rhabdovirus vaccines including VSVΔG and RABVΔG. The glycoprotein (G) of SAD L16 is ideal in order to infect tonsil cells, noting that tonsils are small organs in the back of the throat. Most preferably, ΔG vectors are pseudotyped with the SAD G or SAD G derivatives, i.e. VSVΔG(SAD-G) or RABVdG(SAD-G) are preferred for oral immunization. The feasibility to pseudotype VSVΔG with SAD G is illustrated in FIG. 10.

In accordance with another preferred embodiment of the second aspect of the invention the use further comprises a boost immunization with the composition of the first aspect of the invention or with virus-like particles presenting the chimeric immunogen in their envelope.

After initial immunization, a boost immunization is a re-exposure to the immunogen. It is intended to increase immunity against that immunogen (e.g. either to reach protective levels, if the first immunization is not sufficient, or restore protective levels, after memory against that antigen has declined through time).

The virus-like particles are preferably as described herein above in connection with the first aspect of the invention.

The boost immunization can be administered intramuscularly (i.m.) or orally. The boost immunization is preferably effected as a repetition of the prime vaccination with VSV and RABV full-length and delta G vectors, including by oral or i.m. route. In addition, prime and boost vaccination can use different application routes and viruses. For example prime vaccination can use i.m. application, while for boost oral route can be used, or vice versa. Similarly, VSV vectors can be used for prime (i.m. or oral), and RABV vectors for boost (i.m. or oral), and vice versa. In a preferred embodiment, when recombinant VSV or delta G VSVΔG pseudotyped with VSV G are used for prime vaccination, boost immunization should employ VSVΔG pseudotyped with the glycoprotein of another rhabdovirus glycoprotein. This stratagem is suitable to circumvent possible interference with boost efficiency by VSV G antibodies generated after prime vaccination.

The present invention relates in a third aspect to the composition of the first aspect for diagnosing a betacoronavirus infection and preferably a SARS-CoV-2 infection in a subject based on a sample, preferably a blood sample and most preferably a serum sample obtained from the subject.

The definitions and preferred embodiments of the above aspects apply mutatis mutandis to the third aspect of the invention.

As is illustrated by FIGS. 5 and 6 the composition of the first aspect of the invention can also be used for diagnostics to identify subjects which had been infected with a betacoronavirus SARS-CoV and have developed antibodies, to assess their immune status.

The present invention relates in a fourth aspect to an immunogenic construct of a betacoronavirus, comprising (i) the spike receptor binding domain (RBD) of a betacoronavirus, wherein the RBD preferably comprises SEQ ID NO: 1 or a variant of SEQ ID NO: 1 being with increasing preference at least 80%, at least 85%, at least 90% and at least 95% identical to SEQ ID NO: 1; C-terminally thereof (ii) a transmembrane anchor being derived from the stem of a rhabodovirus glycoprotein, and preferably derived from members of the Lyssavirus genus, more preferably derived from the rabies (RABV) vaccine strain SAD B19 (molecular clone SAD L16), and most preferably comprising or consisting of the membrane proximal part of the G ectodomain, the trans-membrane and the cytoplasmic tail of the RABV SAD L16 G protein; and (iii) at the N-terminus a signal peptide that promotes high-level translation into the endoplasmatic reticulum.

The definitions and preferred embodiments of the above first three aspects apply mutatis mutandis to the fourth aspect of the invention. In particular the components (i) to (iii) the immunogenic construct of a betacoronavirus are described in greater detail herein above in connection with the first aspect of the present application and these definitions and preferred embodiments apply mutatis mutandis to the fourth aspect of the invention.

The immunogenic construct of a betacoronavirus of the fourth aspect is particularly advantageous in order to elicit an anti-betacoronavirus immune response because (i) the spike receptor binding domain (RBD) of a betacoronavirusis is particularly suitable as immunogen for the generation of a strong immune response and (ii) the transmembrane anchor results in the presentation of the immunogen on the surface of infected cells as well as on virus particles and virus like particles which further enhances the strength of the immune response. Finally, the signal peptide (iii) helps to ensure that the immunogen is effectively presented on the cell surface of infected cells.

The immunogenic construct of a betacoronavirus of the fourth aspect does not occur in nature and therefore may be designated as an “engineered” or “chimeric” immunogen. As discussed above, compared to a natural immunogen, the immunogenic construct of the fourth aspect is optimized or improved with respect to the initiation of an immune response against a betacoronavirus, in particular SARS-CoV2.

In accordance with a preferred embodiment of the fourth aspect of the invention, the signal peptide is derived from an immunoglobulin, preferably from IgG and most preferably from the heavy chain of IgG.

Also such a signal peptide has been described in greater detail herein above in connection with the first aspect of the present application.

In accordance with another preferred embodiment of the fourth aspect of the invention the immunogenic construct consists of less than 500 amino acids, preferably less than 400 amino acids, more preferably less than 380 amino acids and most preferably of less than 370 amino acids.

Also such an immunogenic construct and in particular immunogenic part (ii) thereof has been described in greater detail herein above in connection with the first aspect of the present application. By using immunogenic constructs within these size ranges efficient expression of the immunogens from a viral vector can be ensured and also the possibility of using more than one, for example, two or three identical copies or copies of different variants of the immunogen encoding nucleic acid sequence for the generation of viral vectors expressing two or three copies of the immunogens.

The immunogenic construct most preferably comprises the amino acid sequence of SEQ ID NO: 16 (“minispike” construct) or is encoded by the nucleotide sequence of SEQ ID NO: 17. The immunogenic construct may also comprise an amino acid sequence being with increasing preference at least 80%, at least 85%, at least 90% and at least 95% identical to SEQ ID NO: 16 or being encoded by a nucleotide sequence being with increasing preference at least 80%, at least 85%, at least 90% and at least 95% identical to SEQ ID NO: 17. Since SEQ ID NO: 1 is comprised in SEQ ID NO: 16, the immunogenic construct may instead of SEQ ID NO: 1 also a variant of SEQ ID NO: 1 as described herein above.

In accordance with a further preferred embodiment of the fourth aspect of the invention the betacoronavirus is selected from SARS-CoV-2, MERS-CoV, SARS-CoV-1, 0043 and HKU1, and is preferably SARS-CoV-2.

As discussed in connection with the first aspect of the invention these five betacoronaviruses are known human pathogens.

In connection with the fourth aspect the present invention also relates to a nucleic acid molecule encoding an immunogenic of the fourth aspect.

The nucleic acid molecule can be DNA or RNA and is preferably DNA.

The present invention relates in a fifth aspect to a virus vaccine vector or a plasmid or a DNA or RNA preparation expressing the immunogenic construct of the fourth aspect of the invention or comprising the nucleic acid molecule of the fourth aspect of the invention.

The definitions and preferred embodiments of the above aspects apply mutatis mutandis to the fifth aspect of the invention.

The virus vaccine vector may be formulated as a viral delivery system for delivering the immunogenic construct or the nucleic acid into a cell. The plasmid or a DNA or RNA preparation virus vaccine vector may be formulated as a nonviral delivery system for delivering the immunogenic construct or the nucleic acid to a cell.

The virus vaccine vector is preferably a recombinant rhabdovirus vector as defined in accordance with the present application.

In accordance with a further preferred embodiment of the fifth aspect of the invention the virus vaccine vector is a RNA virus vaccine vector, preferably a measles virus or parainfluenza virus (PIV), or a DNA virus vaccine vector, preferably a Modified-Vaccinia-Ankara-Virus (MVA), Adenovirus or Adenoassociated virus (AAV).

Viral vectors are tools commonly used by molecular biologists to deliver genetic material into cells. This process can be performed inside a living organism (in vivo) or in cell culture (in vitro). Viruses have evolved specialized molecular mechanisms to efficiently transport their genomes inside the cells they infect. RNA virus vaccines are derived from RNA viruses and DNA virus vaccines are derived from DNA viruses.

Non-limiting examples of RNA virus vaccine vectors are measles virus or parainfluenza virus (PIV). Measles virus belongs to the Paramyxovirus family. Infection confers lifelong immunity. Human parainfluenza viruses (HPIVs) are the viruses that cause human parainfluenza. HPIVs are a paraphyletic group of four distinct single-stranded RNA viruses belonging to the Paramyxoviridae family.

Non-limiting examples of DNA virus vaccine vectors are Modified-Vaccinia-Ankara-Virus (MVA), Adenovirus or Adeno-associated virus (AAV). The Modified Vaccinia Ankara-Virus (MVA) is an attenuated vaccine virus of a poxvirus. Adenoviral vectors have been one of the most widely used viruses in gene delivery. They are non-enveloped, double-stranded (ds) DNA viral vectors with a packaging capacity of approximately 35 kb. Over 50 different adenoviral serotypes exist, grouped into six species. AAV are small viruses that infect humans and some other primate species. They belong to the genus Dependoparvovirus, which in turn belongs to the family Parvoviridae. These viruses can insert genetic material at a specific site on human chromosome 19 with near 100% certainty.

Regarding the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims.

The figures show.

FIG. 1—Schematic illustration of the presentation of a betacoronavirus immunogen simultaneously on the surface of infected cells (item 1) and on the surface of virus-like particles (item 2).

FIG. 2—(A) Schematic representation of SARS-CoV-2 Spike protein, and chimeric rhabdovirus minispike containing the RBD of SARS-CoV-2 Spike. (B) Sequence of rhabdovirus minispike. Signal sequence and transmembrane sequence is underlined, linker in italics, and SARS-nCoV-2 residues in bold letters.

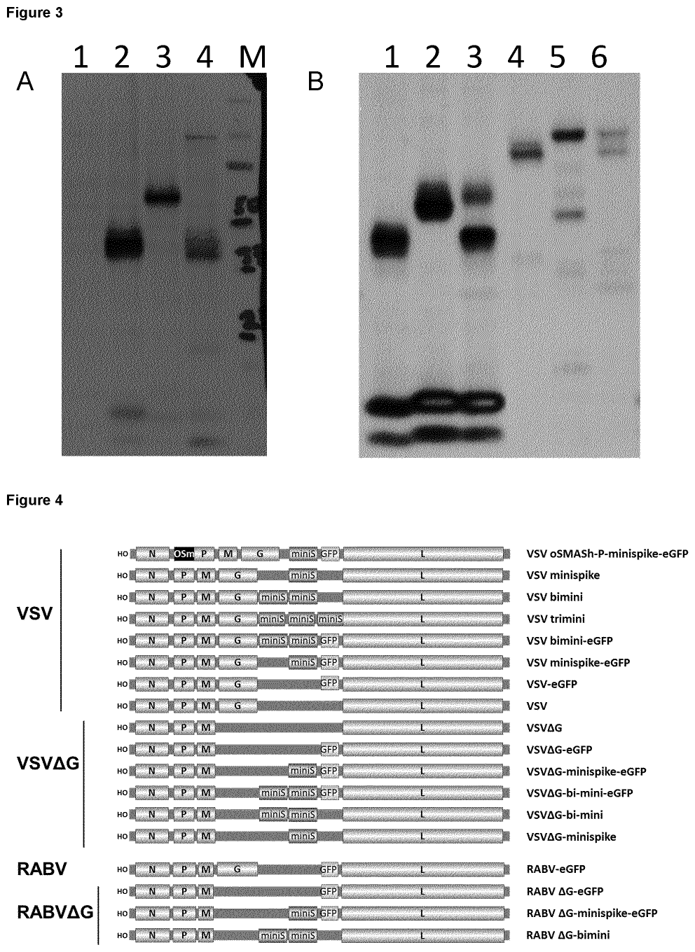

FIG. 3—(A) Expression of minispikes in HEK293T cells transfected with pCAGGs-minispike (lane 2) and in BHK-21 cells infected with VSVΔG-minispike-eGFP (lane 4). Lane 1 shows irrelevant vector transfection, lane 3 HEK293T cells transfected with the pCAGGS-RABV G as a positive control for the peptide serum recognizing the C-tail of minispike and G. Bands of heterogenous size indicate glycosylation of the minispike. Lane 4 shown minispike expressed from recombinant VSVdG minispike (B) Complex glycosylation of minispike protein in HEK293T cells transfected with pCAGGs-minispike (lanes 1-3). Extracts were treated with PNGase F, which cleaves off all N-linked oligosaccharides (lane 1), or left untreated (lane 2) or treated with Endo H, cleaving off N-linked mannose rich oligosaccharides, but not highly processed complex oligosaccharides. Lanes 4-6 show RABV G treated in parallel (4: PNGaseF, 5, untreated, 6: EndoH)

FIG. 4—Schematic illustration of recombinant VSV and RABV virus constructs used here.

FIG. 5—VSVdG-minispike eGFP-infected BHK-21 are recognized by reconvalescent COVID-19 patient sera. (A) Infected unfixed cell cultures were incubated with the indicated patient sera, followed by incubation with fluorescent anti-human IgG antibodies (upper panel). Lower panel shows eGFP fluorescence in cell cultures (vector control). (B) COVID-19 patient sera recognize minispike protein in acetone-fixed cell cultures. ELISA-positive serum recognizes minispike expressing cells (left image). Serum antibodies were stained with anti-human IgG secondary antibodies labelled with Alexa555.

FIG. 6—VSVdG-bi-minispike (VSVdG-bimini) infected cells are recognized by reconvalescent COVID-19 patient sera. BHK-21 cells were infected for 16 hours with VSVdG-bimini virus and after acetone fixation incubated with COVID-19 patient sera (#1-4, 9-10) or sera from healthy persons (#5, 6, 8), followed by anti-human IgG-Alexa488 (green fluorescence).

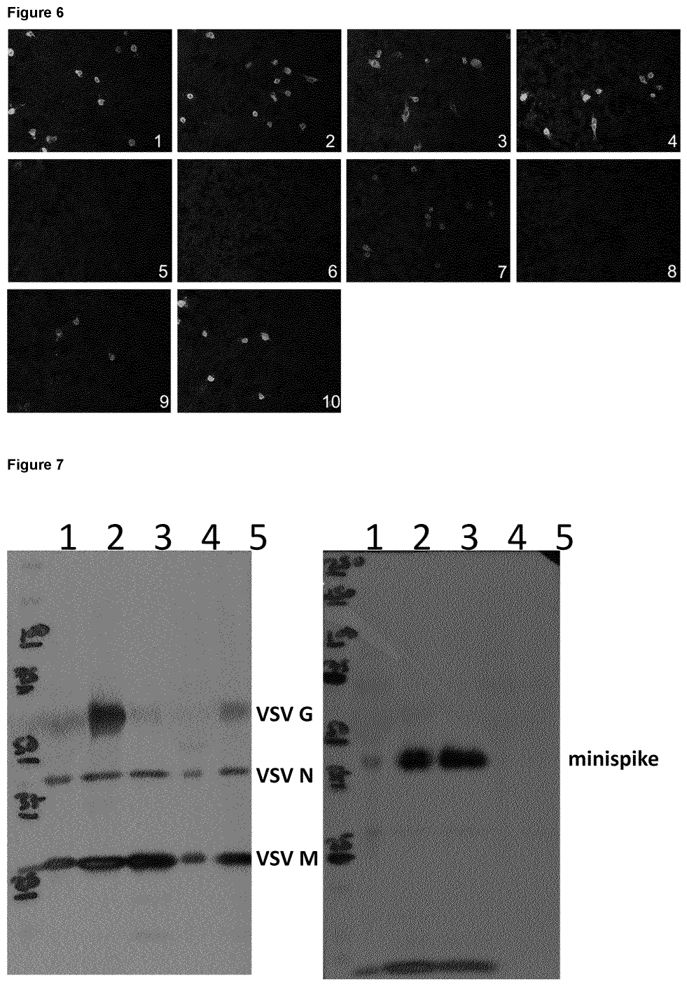

FIG. 7—Incorporation of minispike protein in VSV virus particles. Cell-free VSVdG-minispike-eGFP particles (lanes 1-3) were generated in HEK293T cells expressing VSV-G from transfected pCAGGS-VSV G and analyzed by Western blot. In lane 1 unconcentrated cell culture supernatant was applied, in lanes 2 and 3 virions which were ultracentrifuge-purified through a sucrose cushion. Lanes 4 and 5 contains stocks of standard VSVdG-eGFP, and of full-length wt VSV-eGFP grown in BHK cells, respectively. Lanes were loaded with 3×10E6 infectious units. Blots were incubated with anti-VSV serum (left panel) and a serum recognizing the RABV G-derived C-tail of the chimeric SARS-CoV-2/rhabdovirus minispike (right panel). Note that efficient incorporation into VSV virus particles is achieved even in the presence of high amounts of competing VSV G protein (lane 2).

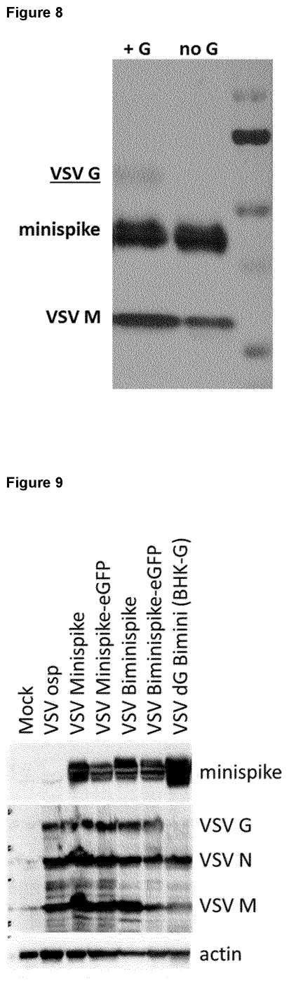

FIG. 8—Preparation of VSVdG Minispike VLPs lacking G. VSVdG-eGFP(VSVG) was used to infect non-G-complementing cells to produce VLPs decorated only with minispike protein. Cell culture supernatant was harvested one day after infection and VLPs purified by ultracentrigugation. VLPs were analyzed by Western blot with HCA-serum to detect minispike protein, and VSV Serum for detection of G protein. In contrast to particles produced in the presence of G (+G), VSV G was not detected in VLPs from non-complementing cells (lane: no G).

FIG. 9—Gene copy number determines expression levels of minispike protein. Lysates from cells infected with the indicated viruses were analyzed by Western blot with HCA-5 serum for expression levels of the minispike protein. Actin and VSV antibodies were used for normalization. Two copies of the minispike gene encoded in full length VSV vector lead to higher expression of the protein. Note that the presence of an unrelated extra gene (eGFP reporter) in general attenuates expression levels. For full length VSV the ranking is therefore: VSV-Bimini>-bimini-eGFP>-minispike>-minispike-eGFP. The VSV delta G version, VSVdG-bimini, encoding 1 gene less than VSV-bimini expresses the highest amounts of minispike.

FIG. 10—Incorporation of minispike protein in RABV virus particles. Spike protein and rhabdovirus G constructs expressed from transfected plasmids were used to pseudotype VSVdG-eGFP and RABV SADdG-eGFP. As predicted, the minispike construct is suitable to produce both VSV and RABV VLPs. In addition, VSVdG can be pseudotyped with RABV G, (left panel) supporting efficient entry into tonsil cells during oral immunization.

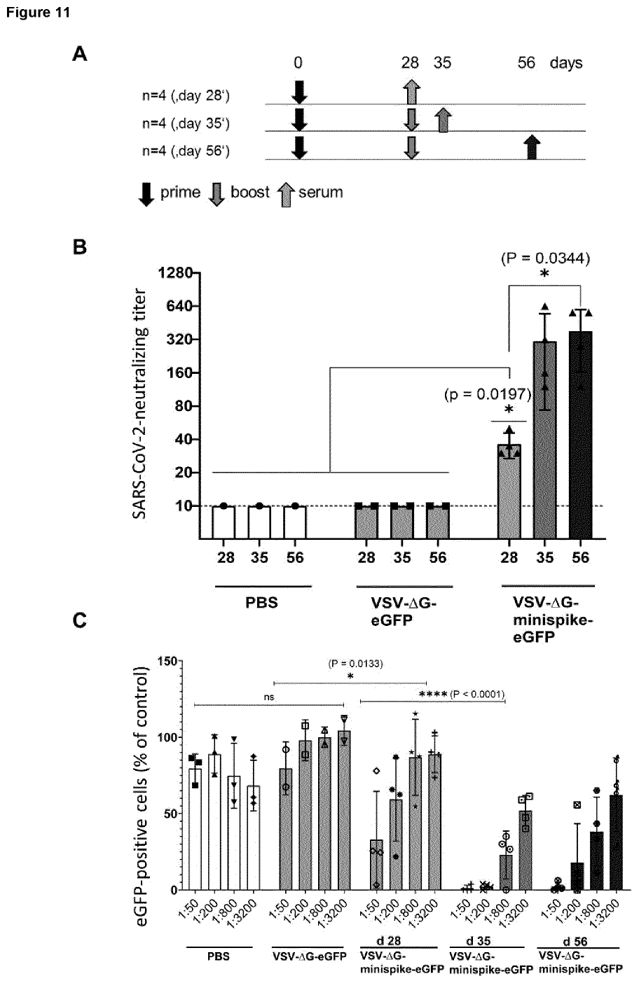

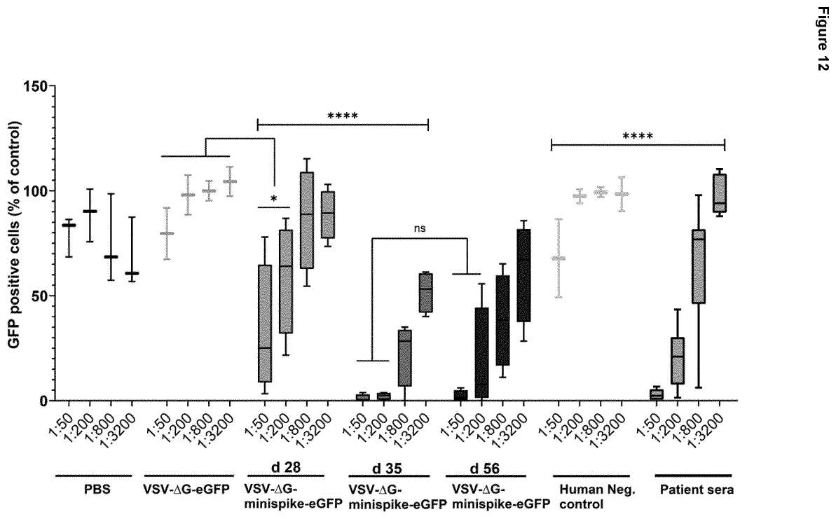

FIG. 11—Immunization with VSVΔG-minispike-eGFP vaccination elicits high levels of SARS-CoV2-neutralizing antibodies in BALB/c mice. (A) Immunization Scheme. BALB/c mice were immunized i.m. with 1×10⁶ infectious units of VSV G-complemented VSVΔG-minispike-eGFP and controls including VSV G-complemented VSVΔG-eGFP, or PBS. Twenty-eight days after immunization serum was collected from 4 vaccinated mice, while 8 mice received an i.m. boost immunization with the same dose of virus. (B) Serum neutralization tests performed with a clinical isolate of SARS-CoV-2. The neutralizing titer of sera from vaccinated and control mice as indicated is expressed as the reciprocal of the highest dilution at which no cytopathic effect was observed. Each point represents data from one animal at the indicated time points. The bars show the mean from each group and the error bars represent standard deviations. Significant neutralizing activity was observed in mice receiving only a prime vaccination (day 28, light blue). A boost immunization further significantly enhanced neutralizing titers (days 35 and 56). (C) Neutralization of VSVΔG(S) pseudotype viruses by individual mouse sera. Mouse sera collected on day 28 (receiving prime immunization only) or at 35 and 56 days (receiving prime and boost immunization) were serially diluted as indicated and analyzed for neutralization VSV(S) pseudotype particles. GFP-encoding pseudotype virions were incubated with increasing dilutions of mouse sera or medium control before infection of VeroE6 cells. The graph shows percentage of GFP-positive cells in relation to medium controls (100%) and in dependence of dilution. Data points represent the average of three technical replicates, bars indicate standard deviation, and statistical significance was determined by one-way ANOVA.

FIG. 12—Similar virus-neutralizing titers in vaccinated mice and COVID-19 patients. VSVΔG(S) neutralization activity of sera from vaccinated mice and human immune sera tested positive for S antibodies by ELISA were compared. The graph shows percentage of GFP-positive cells in relation to medium controls and in dependence of dilution. ELISA-positive human sera revealed VSV(S)-neutralizing activity and are included in the grey boxes showing activity at the indicated dilutions. Primed mice (d28) exhibited neutralizing activity almost comparable to those of human patients, while boosted mice (d35 and d56) exhibited superior activity. Bottom and top of each box represent the first and third quartiles respectively. Whiskers represent the lowest and highest data points of the lower and upper quartile respectively. Student's t-test and One-way ANOVA were performed to determine statistical significance.

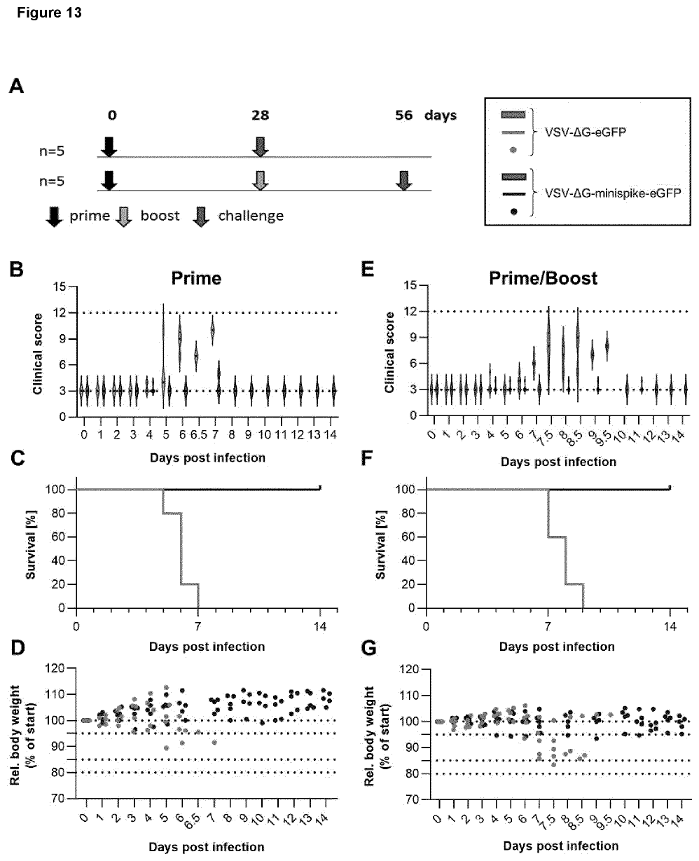

FIG. 13—Transgenic mice are protected from SARS-CoV-2-induced respiratory disease after a single immunization with VSVΔG-minispike-eGFP. (A) Immunization and challenge schematic. C57BL/6 K18-hACE2 mice (5 per group) were immunized (1×106 ffu intramuscularly) once (prime, black arrow) or twice (boost, grey arrow) four weeks apart with either VSV-ΔG-minispike-eGFP (indicated in blue in panels B-G) or VSV-ΔG-eGFP (indicated in red in panels B-G) and challenged with 1×104 TCID50 SARS-CoV-2 (Wetzlar isolate) administered intranasally four weeks after the last immunization. Mice were monitored daily for development of disease for 14 days. (B-D) Evaluation of clinical disease of challenge after prime immunization. (E-G) Evaluation of clinical disease of challenge after prime/boost immunization. (B and E) Clinical score development assessed by body weight loss, general appearance, and behavior. 3: healthy; 4-6: mild disease; 7-9: severe disease; 10-12: moribund. (C and F) Survival plots. (D and G) Body weights of individual mice relative to the weight at challenge infection. Dotted lines indicate limits of clinical scores (>95%: score=1, 85-95%: score=2; 80-85%: score=3; <80%: score=4).

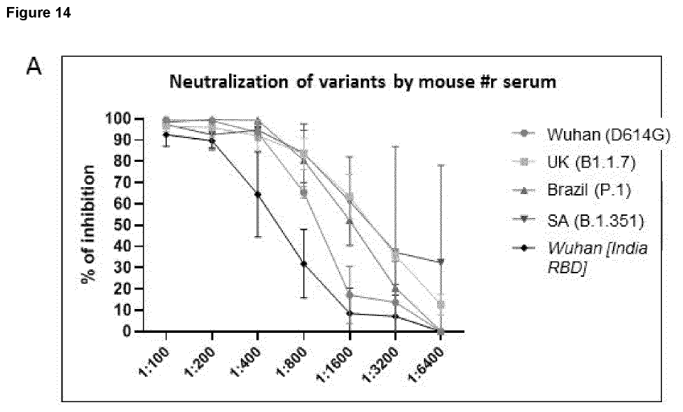

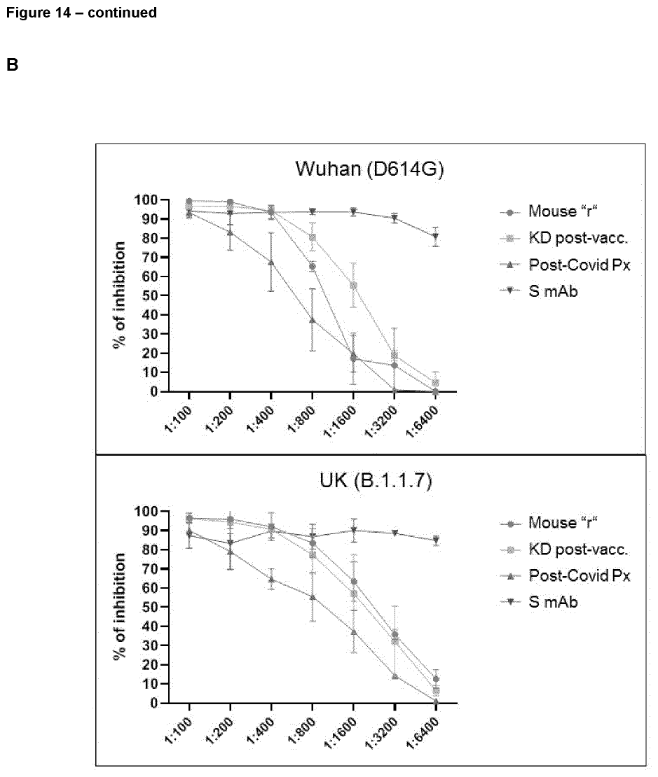

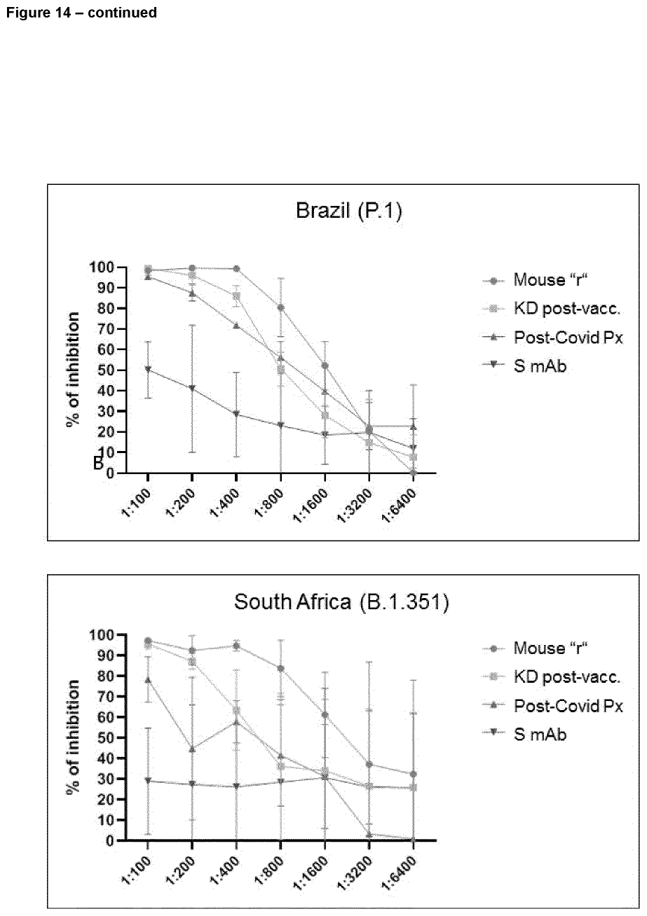

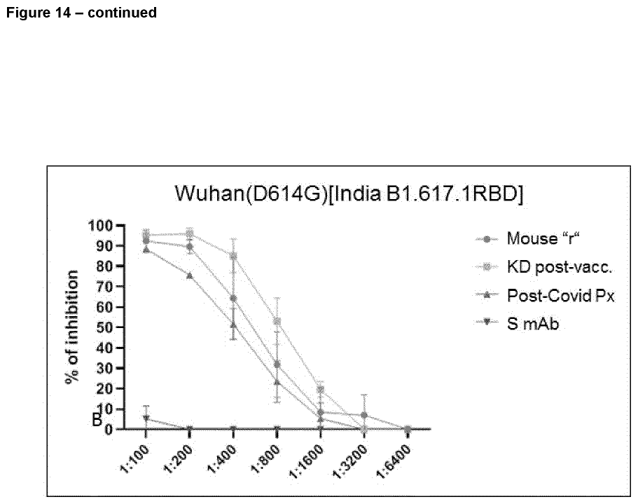

FIG. 14—Neutralization of variants of concern (VOC) by sera from VSVΔG-minispike-eGFP vaccinated mice. (A) VSVΔG pseudotype viruses carrying the indicated S proteins from emerging SARS-CoV-2 variants were incubated with serum from a vaccinated mouse (#r, 56d) and checked for infectivity of VeroE6 cells. All viruses carrying natural variant S proteins, including SA (B.1.351) were neutralized effectively (IC50>1:800), while an artificial S protein (Wuhan(D614G) with Indian RBD sequence) revealed a slightly lower susceptibility (IC50>1:600). (B) Comparison of neutralizing activity of sera from vaccinated mouse (mouse “r”, BNT162-b2 double vaccinated subject (KD post vacc.), convalescent COVID-19 patient (Post-Covid Px), and an S monoclonal antibody (SmAb). Data are from three independent experiments.

The examples illustrate the present invention.

### EXAMPLE 1—DESIGN OF RHABDOVIRUS MINISPIKE

The sequence of SARS-CoV-2 (Severe acute respiratory syndrome coronavirus 2 isolate Wuhan-Hu-1, complete genome, NCBI Reference Sequence: NC_045512.2) was used to prepare a synthetic human codon optimized DNA spanning the entire RBD coding sequence of the S gene and a few flanking residues (residues 314-541, QTSN . . . KCVNF, (SEQ ID NOs 24 and 25)) (i.e. different from RBD constructs described in Wrapp et al. 2020, and Tai et al., 2020). An upstream signal peptide derived from human IgG (Ig G HV 3-13) was directly added to promote high-level translation into the ER. At the C-terminus the RBD was fused via a short synthetic linker (GSGS, (SEQ ID NO: 26)) to a transmembrane/stem anchor derived from RABV SAD L16 G protein, containing the membrane proximal part (stem) of the G ectodomain, trans-membrane, and cytoplasmic tail of SAD G. The entire construct contains 367 amino acid residues, including the signal sequence that is cleaved off during translation (+1 stop codon) (FIG. 2).

The RABV G transmembrane/stem anchor is identical to that described previously where it was successfully used to mediate surface expression and rabies virus incorporation of a dsRed fusion protein (Klingen, Conzelmann, & Finke, 2008). While the RABV stem construct does not contain N-glycosylation sites, two putative N-Glycosylation sites are present in the RBD part of the minispike at 2 nearby positions (NITNLCPFGEVFNAT (SEQ ID NO: 27)), which might support correct transport and folding of the chimeric protein and facilitate analysis of the protein (FIG. 2).

Expression of the minispike construct in HEK293T cells from transfected pCR3-minispike was analyzed in Western blot with an anti-RABV C-tail peptide serum, HCA-5 (FIG. 3A, lane 2). The expressed minispike proteins were of the predicted size range, and showed variability in migration, suggesting the presence of differently glycosylated and non-glycosylated minispike species in the whole cell lysates. N-glycosylation and complex N-glycosylation of the minispike protein was verified by PNGase F and EndoH digestion, respectively (FIG. 3B). This indicated successful transport of the chimeric minispike protein through the ER and Golgi apparatus.

### EXAMPLE 2—GENERATION OF MINISPIKE-EXPRESSING VSVs AND RABVs

To obtain recombinant VSVΔG and full-length replicating-competent rVSV encoding the minispike construct, a plasmid clone, pVSV-eGFP, of vesicular stomatitis Indiana virus (VSIV)(Lawson et al., 1995; M. J. Schnell et al., 1998) was first used to exchange the VSV G ORF with the minispike ORF, to yield pVSV ΔG minispike-eGFP. Full length pVSV-minispike-eGFP was generated by exchange of the eGFP cassette with a minispike-eGFP cassette form the ΔG construct.

The viral Minispike constructs were then used to delete eGFP to yield viruses lacking the reporter gene, pVSVΔG-minispike, and VSV-minispike, respectively. VSV and VSVdG expressing two copies of the minispike were generated by replacing eGFP with a second copy of the minispike gene to yield VSVΔG-bi-minispike and VSV-bi-minispike, or by insertion of an extra minispike gene for VSVdG-bi-mini-eGFP and VSV bi-mini-eGFP.

VSV rescue from cDNA was performed in HEK293T cells transfected with the viral cDNA plasmids directing T7 RNA polymerase-driven transcription of viral antigenome RNAs along with expression plasmids encoding T7 RNA polymerase and VSV helper proteins N, P, and L (pCAG-T7, -N, -P, -L; all from addgene).

RABVΔG-eGFP expressing the minispike gene was performed as described previously (Ghanem, Kern, & Conzelmann, 2012; Wickersham, Finke, Conzelmann, & Callaway, 2007).

The organization of recombinant VSV and RABV viruses is shown in FIG. 4

### EXAMPLE 3—VSV-EXPRESSED MINISPIKE IS PRESENTED AT THE CELL SURFACE AND RECOGNIZED BY COVID-19 PATIENT SERA

Following virus rescue, VSVΔG-minispike was used to infect BHK-21 to verify expression of virus-encoded minispike protein (FIG. 3, lane 4). Again, differently sized glycosylated minispike proteins were detected in the cell lysates by Western blot using the RABV G tail serum.

To examine correct conformation of the RBD domain in the chimeric minispikes, BHK-21 Cells infected at a low MOI with VSVdG-minispike or VSVdG-bi-mini were probed with sera from COVID-19 patients previously tested positive by ELISA in an accredited diagnostics institute. At a dilution of 1:300, the sera from positive patients recognized infected cells, as determined by Alexa-555 labeled anti-human IgG antibodies, while no signal was obtained with a COVID-19 negative sera. Anti-RABV C-tail serum and eGFP immune fluorescence was used to discriminate virus-infected cells. Minispike expressing cells were similarly recognized by the sera both after fixation with acetone, and in live cells without fixation (FIG. 5, panels A and B, respectively). Cells expressing minispike from the VSVΔG-bimini virus were also recognized (FIG. 6)

To corroborate that staining was caused by SARS-CoV-2 specific antibodies, and not by VSV-specific antibodies, co-infections with VSVΔG minispike-eGFP and VSVΔG-tag-BFP was performed. Exclusively green fluorescent cells were recognized by the patient IgG antibodies, while blue fluorescent cells were not (not shown). These experiments revealed that the minispike protein expressed from recombinant rhabdoviruses displays a conformation which is recognized by natural antibodies made in response to SARS-CoV-2 infection and COVID-19 disease, and that it folds into a structure analogous to that of the natural RBD of SARS-CoV-2. Rhabdovirus vectors expressing minispike from one or more genes are thus suitable as a COVID-19 vaccine.

### EXAMPLE 4—MINISPIKE-PSEUDOTYPE VIRUSES AND VIRUS-LIKE PARTICLES (VLPS)

As minispike proteins expressed from VSVΔG contain the VSV envelope-compatible RABV G membrane anchor and C-tail, it was expected that minispikes are incorporated into VSV particles generated in the infected cells. To verify this, VSVΔG-minispike stocks were generated in VSV-G expressing HEK293T cells and concentrated over a sucrose cusion by ultracentrifugation (FIG. 7). For preparation of one of the stocks, VSV G was expressed only 6 hours before VSVΔG infection, while for the other, VSV-G was allowed to accumulate to high levels for 24 hours, before infection. Viruses were harvested 24 hours post infection, followed by ultracentrifugation. Equivalent infectious units (10E6) viruses were processed for Western blot analysis and blots were analyzed with a whole VSV serum (FIG. 7A), and anti-RABV C-tail serum (FIG. 7B) to detect the RABV-derived part of the minispike. In all minispike-encoding virion preparations was minispike protein detected, revealing efficient incorporation, likely as a trimer, into the viral membrane (FIG. 7B, lanes 1-3). Of note, even substantial overexpression of VSV G could not prevent minispike incorporation into virions (compare lanes 2 and 3), revealing strong and competitive incorporation into VSV VLPs.

To demonstrate that minispike alone, i.e. in the absence of G is sufficient to generate minispike-decorated, and non-infectious VLPs, VSVdG-minispike-eGFP was grown in non-G-complementing cells, and particles present in the cell culture supernatant harvested as described before. Indeed, in contrast to particles generated in G-transfected cells, pure VLPs lacking G were produced in non-complementing cells (FIG. 8).

### EXAMPLE 5—MINISPIKE GENE COPY NUMBER DETERMINES LEVEL OF EXPRESSION

To verify that additional copies of the minispike gene in the genome of VSV vectors lead to enhanced expression of the protein, we constructed VSVs encoding two minispike genes in tandem (VSV-bimini, or VSV-bi-minispike) in the presence or absence of a eGFP reporter gene (see FIG. 4 for genome organization). Viable viruses were readily rescued from plasmids. To compare immunogen expression of bimini-vectors and corresponding VSV containing only a single minispike gene, cells were infected in parallel and minispike expression was analyzed by Western blotting. Indeed, the double gene dose vectors led to accumulation of higher minispike protein levels compared to the single gene dose vectors. As expected, the presence of the extra eGFP gene in the VSV vectors (7 or 8 genes encoded in total) reduced minispike expression in both single and double minispike gene dose VSVs. The highest expression was obtained in VSVdG-bimini lacking the eGFP gene, as this vector encodes only 6 genes in total.

### EXAMPLE 6—INCORPORATION OF MINISPIKE PROTEIN IN RABV VIRUS PARTICLES

The design of the minispike protein has the advantage that upon expression from recombinant vectors both VSV and RABV particles and VLPs are generated. To illustrate incorporation into RABV particles, different SARS-CoV-2 Spike protein constructs, minispike, and RABV SAD G protein were expressed from transfected plasmids and used to pseudotype VSVdG-eGFP and RABV SADdG-eGFP. As predicted, the minispike construct is suitable to produce both VSV and RABV VLPs. Of note, the minispike is incorporated into the envelope of RABVdG as efficiently or even better as the authentic SAD G (FIG. 10). Successful complementation of VSVdG with RABV SAD G (left panel) illustrated also the suitability of SAD G-pseudotyped VSV vectors for infection of tonsil cells and oral immunization.

### EXAMPLE 7—IMMUNIZATION WITH VSVΔG-MINISPIKE-EGFP ELICITS HIGH LEVELS OF SARS-CoV2-NEUTRALIZING ANTIBODIES IN BALB/C MICE

To assess the suitability and the sufficiency of a single round VSVΔG minispike replicon to elicit an immune response, BALB/c mice were immunized with VSVΔG-minispike-eGFP (G) by intramuscular (i.m.) administration. Virus stocks were produced under limiting VSV G complementation, i.e. only 6 hrs of VSV G expression, to prevent excess formation of non-viral G vesicles. Four mice received a single dose of 1×10⁶ infectious particles, while 8 mice received an additional boost with the same virus preparation and dose 28 days following prime vaccination. As controls, mice immunized the same way with VSVΔG-eGFP (VSV G) (n=2 for each condition) or with PBS (n=1 for each condition) were used. The 4 mice receiving only prime vaccination were sacrificed at day 28, and 4 boosted mice each at day 35 (n=4) and day 56 (n=4), to collect serum (FIG. 11A).

Virus neutralization assays were performed with a SARS-CoV-2 virus isolate from Wetzlar, Germany. Notably, all 4 mice immunized only once developed detectable titers of SARS-CoV-2 neutralizing antibodies in the range of 1:20-1:40 dilutions. Boost vaccination further increased neutralizing titers to 1:160-1:640 (FIG. 11B).

For verification of the notable neutralizing titers after prime immunization in an independent assay, we also produced VSV particles pseudotyped with a functional S protein, VSV-eGFP-ΔG-GLuc (SΔC19). Neutralization assays with the VSV pseudotype viruses confirmed the induction of significant levels of S-neutralizing antibodies in mice receiving a single prime vaccination and further enhancement of neutralization activity by boost immunization (FIG. 11C).

To directly compare the neutralizing activities of sera from vaccinated mice and from COVID-19 patients, VSV-eGFP-ΔG-GLuc (SΔC19) neutralization assays were employed. Most intriguingly, the group of mice immunized only once (boxes labeled d 28 in FIG. 12), developed neutralizing antibodies with a capacity almost equal to those of the group of COVID-19 patients, illustrating a powerful induction of humoral immunity by vaccination with the single round VSVΔG-minispike-eGFP replicon. Boost immunization further enhanced neutralizing titers to exceed those of patients (FIG. 12).

These results illustrate that a small antigen, the RBD of SARS-CoV-2, if presented in the form of the present chimeric minispike protein from a safe, spreading-deficient single round biosafety level 1 rhabdovirus replicon is sufficient to elicit high levels of neutralizing antibodies.

### EXAMPLE 8—K18-HACE2 MICE ARE PROTECTED FROM SARS-COV-2-INDUCED RESPIRATORY DISEASE AFTER A SINGLE IMMUNIZATION WITH VSVΔG-MINISPIKE-EGFP

To assess the protective capacity of the VSVΔG-minispike-eGFP vaccine we used transgenic K18-hACE2 C57BL/6 mice, which were previously shown to develop respiratory disease resembling severe COVID-19 (Yinda et al., 2021). Five mice each were immunized as before with VSVΔG-minispike-eGFP or VSVΔG-eGFP control and challenged intranasally with 10⁴ TCID50 of SARS-CoV-2 Wetzlar, either following prime immunization or homologous boost immunization (FIG. 13A). Mice immunized with the VSVΔG-eGFP control developed respiratory disease beginning as early as day 5 after infection (FIGS. 13 B and E), which progressed over the following 3-4 days, and animals ultimately succumbed to disease 6-9 days after infection (FIG. 13 C,F). These animals lost only approximately 10-15% of their initial weight (FIG. 13 D,G), which indicates that they experienced a largely respiratory syndrome. In contrast, mice immunized with VSVΔG-minispike-eGFP experienced no clinical signs of disease (FIG. 13 B, E), and all animals survived the infection (FIG. 13C,F) with little to no weight loss during the study (FIG. 13 D, G). This demonstrates the protective power of the VSVΔG-minispike-eGFP replicon vaccine, since a single immunization prevented the development of lethal COVID-19 respiratory disease.

### EXAMPLE 9—ANTIBODIES ELICITED IN RESPONSE TO VSVΔG-MINISPIKE-EGFP VACCINATION NEUTRALIZE VARIANTS OF CONCERN

The simultaneous presentation of distinct RBD antigenic sites is of relevance not only for the efficiency of a vaccine against the homologous virus, but also in the light of emergence and spread of SARS-CoV-2 variants of concern (VOC). Several mutated SARS-CoV-2 strains appeared at the end of 2020 and rapidly expanded to become the dominant strains. Recently emerged VOCs include British/UK (B.1.1.7), South Africa (B.1.351; 501Y.V2), and Brazil (P.1) variants, which have acquired 9, 10, and 12 mutations, respectively, in the S protein gene, facilitating transmission and spread of the virus, or reduce its sensitivity to vaccine-induced or therapeutic antibodies (Baum et al., 2020; Weisblum et al., 2020). Mutations in the ACE2 binding RBD surface are of greatest concern because the neutralizing antibody response predominately targets this region. The B.1.1.7 RBD contains the single N501Y mutation. B.1.351 has three changes in the RBD (K417N, E484K, and N501Y) and P.1 has a very similar triplet mutation (K417T, E484K, and N501Y) which confer increased affinity for ACE2. Neutralization of B.1.351 by both patient and vaccine-induced induced antibodies is reduced, and for the BioNTech/Pfizer vaccine a 9-fold reduction in neutralization was reported (Zhou et al., 2021). Despite similar RBD mutations, P.1 is less resistant to neutralization, suggesting that changes outside the receptor-binding domain (RBD) can also affect neutralization (Dejnirattisai et al., 2021).

To assess whether the RBD antibodies generated after VSV-ΔG-minispike immunization are active against the VOCs, mouse sera were used for neutralization assays with VSV pseudotyped with Spike variants from the prevalent Wuhan virus carrying a stabilizing D614G mutation (Wuhan (D614G), UK (B.1.1.7), Brazil (P.1.), and South-Africa (B.1.351) virus variants, as well as a chimeric spike construct (Wuhan(D614G) with RBD from India B.1.617.1), which does not occur in nature so far. Notably, serum from vaccinated mice neutralized all virus variants, including viruses with the South African spike protein (B.1.371) (FIG. 14A). B.1.351 is currently is the variant of greatest concern, since an approved vaccine (ChAdOx, Vaxzevria from Astra Zeneca) fails to protect against this variant patients previously infected with the ancestral Wuhan virus variant are not protected against B.1.351 infection and disease. Fortunately, however, other vaccines like BNT162b2 (Comirnaty; BioNTech/Pfizer) or mRNA-1273 (Moderna Biotech) are protective (Edara et al., 2021; Muik et al., 2021; Xie et al., 2021). Notably, neutralizing titers of VSVΔG-minispike-eGFP immunized mice against the South-Africa variant B.1.351 exceeded those from a BNT162b2-vaccinated representative patient serum (FIG. 14B) indicating protection of the minispike against the VOC B.1.351. Neutralizing titers for UK B.1.1.7, Brazil P.1, and the chimeric UK/India variants were similar for minispike-immunized mice and BNT162b2-vaccinated persons, indicating protection against these variants as well. As indicated before (Supasa et al., 2021), neutralizing titers from convalescent sera were lowest for all variants. The results show that immunization with a minispike comprising the authentic WuhanRBD sequence as described here, elicits antibodies able to neutralize emerging SARS-CoV-2 variant.

### EXAMPLE 10—CONSTRUCTION OF MINISPIKES OF SARS-COV-2 VARIANTS

The Wuhan minispike encoded by VSVΔG-minispike-eGFP elicited neutralizing antibodies against the original SARS-CoV-2 (Wuhan) as well as VSV pseudotypes carrying spike proteins of variants of concern. Anyway, it might be advantageous to modify the VSVΔG-minispike replicon vaccine to encode minispikes derived from variants in order to achieve even broader protection. Accordingly, minispike constructs were produced possessing the mutations of the most important variants of concern:

pCR3 Minispike [E484K], [E484K, N501Y], [K417N, E484K], [K417N, E484K, N501Y] (corresp. to South Africa, B.1.351), [K417T, E484K, N501Y] (corresp. to Brazil, P.1), [L452R, E484K, N501Y], [L452R] (corresp. to Cal.20C), [L452R, E484Q] (corresp. to B.1.617.1), and [L452R;T478K] (corresp. to B.1.617.2) and the corresponding sequences inserted into VSVΔG-minispike-eGFP.

In addition to VSVΔG replicons encoding individual minispike variants, replicons encoding two different minispikes were generated, e.g. VSVΔG-minispike[Wuhan]+minispike[E484K]-eGFP.

### REFERENCES

Abraham, G., & Banerjee, A. K. (1976). Sequential transcription of the genes of vesicular stomatitis virus. Proc Natl Acad Sci USA, 73(5), 1504-1508.
Astori, M., & Kraehenbuhl, J. P. (1996). Recombinant fusion peptides containing single or multiple repeats of a ubiquitous T-helper epitope are highly immunogenic. Mol Immunol, 33(13), 1017-1024. doi:10.1016/50161-5890(96)00068-5
Baum, A., Fulton, B. O., Wloga, E., Copin, R., Pascal, K. E., Russo, V., . . . Kyratsous, C. A. (2020). Antibody cocktail to SARS-CoV-2 spike protein prevents rapid mutational escape seen with individual antibodies. Science. doi:10.1126/science.abd0831
Bishnoi, S., Tiwari, R., Gupta, S., Byrareddy, S. N., & Nayak, D. (2018). Oncotargeting by Vesicular Stomatitis Virus (VSV): Advances in Cancer Therapy. Viruses, 10(2). doi:10.3390/v10020090
Black, B. L., & Lyles, D. S. (1992). Vesicular stomatitis virus matrix protein inhibits host cell-directed transcription of target genes in vivo. J Virol, 66(7), 4058-4064.
Buchholz, U. J., Bukreyev, A., Yang, L., Lamirande, E. W., Murphy, B. R., Subbarao, K., & Collins, P. L. (2004). Contributions of the structural proteins of severe acute respiratory syndrome coronavirus to protective immunity. Proc Natl Acad Sci USA, 101(26), 9804-9809. doi:10.1073/pnas.0403492101
Conzelmann, K.-K. (2013). Reverse Genetics of Mononegavirales: The Rabies Virus Paradigm. In Y. Nagai (Ed.), Sendai Virus Vector: Advantages and Applications (pp. 1-20). Tokyo: Springer Japan.
Corbett, K. S., Edwards, D. K., Leist, S. R., Abiona, O. M., Boyoglu-Barnum, S., Gillespie, R. A., . . . Graham, B. S. (2020). SARS-CoV-2 mRNA vaccine design enabled by prototype pathogen preparedness. Nature. doi:10.1038/s41586-020-2622-0
Dagan, N., Barda, N., Kepten, E., Miron, O., Perchik, S., Katz, M. A., . . . Balicer, R. D. (2021). BNT162b2 mRNA Covid-19 Vaccine in a Nationwide Mass Vaccination Setting. N Engl J Med. doi:10.1056/NEJMoa2101765
Dejnirattisai, W., Zhou, D., Supasa, P., Liu, C., Mentzer, A. J., Ginn, H. M., . . . Screaton, G. R. (2021). Antibody evasion by the P.1 strain of SARS-CoV-2. Cell. doi:10.1016/j.cell.2021.03.055
Edara, V. V., Norwood, C., Floyd, K., Lai, L., Davis-Gardner, M. E., Hudson, W. H., . . . Suthar, M. S. (2021). Infection- and vaccine-induced antibody binding and neutralization of the B.1.351 SARS-CoV-2 variant. Cell Host Microbe, 29(4), 516-521.e513. doi:10.1016/j.chom.2021.03.009
Fathi, A., Dahlke, C., & Addo, M. M. (2019). Recombinant vesicular stomatitis virus vector vaccines for WHO blueprint priority pathogens. Hum Vaccin Immunother, 15(10), 2269-2285. doi:10.1080/21645515.2019.1649532
Fehr, T., Bachmann, M. F., Bucher, E., Kalinke, U., Di Padova, F. E., Lang, A. B., . . . Zinkernagel, R. M. (1997). Role of repetitive antigen patterns for induction of antibodies against antibodies. J Exp Med, 185(10), 1785-1792. doi:10.1084/jem.185.10.1785
Garcia-Beltran, W. F., Lam, E. C., St. Denis, K., Nitido, A. D., Garcia, Z. H., Hauser, B. M., . . . Balazs, A. B. (2021). Multiple SARS-CoV-2 variants escape neutralization by vaccine-induced humoral immunity. Cell, 184(9), 2372-2383.e2379. doi:https://doi.org/10.1016/j.cell.2021.03.013
Ghanem, A., & Conzelmann, K. K. (2016). G gene-deficient single-round rabies viruses for neuronal circuit analysis. Virus Res, 216, 41-54. doi:10.1016/j.virusres.2015.05.023 Ghanem, A., Kern, A., & Conzelmann, K. K. (2012). Significantly improved rescue of rabies virus from cDNA plasmids. Eur. J. Cell Biol., 91(1), 10-16.
Hangartner, L., Zinkernagel, R. M., & Hengartner, H. (2006). Antiviral antibody responses: the two extremes of a wide spectrum. Nat Rev Immunol, 6(3), 231-243. doi:10.1038/nri1783
Hoffmann, M., Arora, P., Groß, R., Seidel, A., Hörnich, B. F., Hahn, A. S., . . . Pöhlmann, S. (2021). SARS-CoV-2 variants B.1.351 and P.1 escape from neutralizing antibodies. Cell, 184(9), 2384-2393.e2312. doi:10.1016/j.cell.2021.03.036
Hoffmann, M., Kleine-Weber, H., Schroeder, S., Kruger, N., Herrler, T., Erichsen, S., . . . Pohlmann, S. (2020). SARS-CoV-2 Cell Entry Depends on ACE2 and TMPRSS2 and Is Blocked by a Clinically Proven Protease Inhibitor. Cell. doi:10.1016/j.cell.2020.02.052
Hoffmann, M., Wu, Y. J., Gerber, M., Berger-Rentsch, M., Heimrich, B., Schwemmle, M., & Zimmer, G. (2010). Fusion-active glycoprotein G mediates the cytotoxicity of vesicular stomatitis virus M mutants lacking host shut-off activity. J Gen Virol, 91(Pt 11), 2782-2793. doi:10.1099/vir.0.023978-0
Houser, K. V., Broadbent, A. J., Gretebeck, L., Vogel, L., Lamirande, E. W., Sutton, T., . . . Subbarao, K. (2017). Enhanced inflammation in New Zealand white rabbits when MERS-CoV reinfection occurs in the absence of neutralizing antibody. PLoS Pathog, 13(8), e1006565. doi:10.1371/journal.ppat.1006565
Jaume, M., Yip, M. S., Kam, Y. W., Cheung, C. Y., Kien, F., Roberts, A., . . . Altmeyer, R. (2012). SARS CoV subunit vaccine: antibody-mediated neutralisation and enhancement. Hong Kong Med J, 18 Suppl 2, 31-36.
Klingen, Y., Conzelmann, K. K., & Finke, S. (2008). Double-labeled rabies virus: live tracking of enveloped virus transport. J. Virol., 82(1), 237-245.
Kopecky, S. A., Willingham, M. C., & Lyles, D. S. (2001). Matrix protein and another viral component contribute to induction of apoptosis in cells infected with vesicular stomatitis virus. J Virol, 75(24), 12169-12181.
Lamers, M. M., Beumer, J., van der Vaart, J., Knoops, K., Puschhof, J., Breugem, T. I., . . . Clevers, H. (2020). SARS-CoV-2 productively infects human gut enterocytes. Science. doi:10.1126/science.abc1669
Lawson, N. D., Stillman, E. A., Whitt, M. A., & Rose, J. K. (1995). Recombinant vesicular stomatitis viruses from DNA. Proc Natl Acad Sci USA, 92(10), 4477-4481.
Liu, L., Wei, Q., Lin, Q., Fang, J., Wang, H., Kwok, H., . . . Chen, Z. (2019). Anti-spike IgG causes severe acute lung injury by skewing macrophage responses during acute SARS-CoV infection. JCI Insight, 4(4). doi:10.1172/jci.insight.123158
Majid, A. M., Ezelle, H., Shah, S., & Barber, G. N. (2006). Evaluating replication-defective vesicular stomatitis virus as a vaccine vehicle. J Virol, 80(14), 6993-7008. doi:10.1128/jvi.00365-06
Matsuura, Y., Tani, H., Suzuki, K., Kimura-Someya, T., Suzuki, R., Aizaki, H., . . . Miyamura, T. (2001). Characterization of pseudotype VSV possessing HCV envelope proteins. Virology, 286(2), 263-275. doi:10.1006/viro.2001.0971
Mebatsion, T. (2001). Extensive attenuation of rabies virus by simultaneously modifying the dynein light chain binding site in the P protein and replacing Arg333 in the G protein. J. Virol., 75(23), 11496-11502.
Mebatsion, T., & Conzelmann, K. K. (1996). Specific infection of CD4+ target cells by recombinant rabies virus pseudotypes carrying the HIV-1 envelope spike protein. Proc Natl Acad Sci USA, 93(21), 11366-11370.
Mebatsion, T., Finke, S., Weiland, F., & Conzelmann, K. K. (1997). A CXCR4/CD4 pseudotype rhabdovirus that selectively infects HIV-1 envelope protein-expressing cells. Cell, 90(5), 841-847.
Moeschler, S., Locher, S., Conzelmann, K. K., Kramer, B., & Zimmer, G. (2016). Quantification of Lyssavirus-Neutralizing Antibodies Using Vesicular Stomatitis Virus Pseudotype Particles. Viruses, 8(9). doi:10.3390/v8090254
Muik, A., Wallisch, A.-K., Sanger, B., Swanson, K. A., Mühl, J., Chen, W., . . . Şahin, U. (2021). Neutralization of SARS-CoV-2 lineage B.1.1.7 pseudovirus by BNT162b2 vaccine-elicited human sera. Science, 371(6534), 1152. doi:10.1126/science.abg6105
Polack, F. P., Thomas, S. J., Kitchin, N., Absalon, J., Gurtman, A., Lockhart, S., . . . Gruber, W. C. (2020). Safety and Efficacy of the BNT162b2 mRNA Covid-19 Vaccine. N Engl J Med, 383(27), 2603-2615. doi:10.1056/NEJMoa2034577
Publicover, J., Ramsburg, E., Robek, M., & Rose, J. K. (2006). Rapid pathogenesis induced by a vesicular stomatitis virus matrix protein mutant: viral pathogenesis is linked to induction of tumor necrosis factor alpha. J Virol, 80(14), 7028-7036.
Puelles, V. G., Lutgehetmann, M., Lindenmeyer, M. T., Sperhake, J. P., Wong, M. N., Allweiss, L., . . . Huber, T. B. (2020). Multiorgan and Renal Tropism of SARS-CoV-2. N Engl J Med. doi:10.1056/NEJMc2011400
Sadoff, J., Le Gars, M., Shukarev, G., Heerwegh, D., Truyers, C., de Groot, A. M., . . . Schuitemaker, H. (2021). Interim Results of a Phase 1-2a Trial of Ad26.COV2.S Covid-19 Vaccine. N Engl J Med. doi:10.1056/NEJMoa2034201
Schnell, M. J., Buonocore, L., Boritz, E., Ghosh, H. P., Chernish, R., & Rose, J. K. (1998). Requirement for a non-specific glycoprotein cytoplasmic domain sequence to drive efficient budding of vesicular stomatitis virus. EMBO J, 17(5), 1289-1296. doi:10.1093/emboj/17.5.1289
Schnell, M. J., Mebatsion, T., & Conzelmann, K. K. (1994). Infectious rabies viruses from cloned cDNA. EMBO J, 13(18), 4195-4203.
Supasa, P., Zhou, D., Dejnirattisai, W., Liu, C., Mentzer, A. J., Ginn, H. M., . . . Screaton, G. R. (2021). Reduced neutralization of SARS-CoV-2 B.1.1.7 variant by convalescent and vaccine sera. Cell. doi:https://doi.org/10.1016/j.cell.2021.02.033
Szomolanyi-Tsuda, E., & Welsh, R. M. (1998). T-cell-independent antiviral antibody responses. Curr Opin Immunol, 10(4), 431-435. doi:10.1016/s0952-7915(98)80117-9
Walker, P. J., Blasdell, K. R., Calisher, C. H., Dietzgen, R. G., Kondo, H., Kurath, G., . . . Nbsp lctv Report, C. (2018). ICTV Virus Taxonomy Profile: Rhabdoviridae. J Gen Virol, 99(4), 447-448. doi:10.1099/jgv.0.001020
Wang, C., Li, W., Drabek, D., Okba, N. M. A., van Haperen, R., Osterhaus, A., . . . Bosch, B. J. (2020). A human monoclonal antibody blocking SARS-CoV-2 infection. Nat Commun, 11(1), 2251. doi:10.1038/s41467-020-16256-y
Weisblum, Y., Schmidt, F., Zhang, F., DaSilva, J., Poston, D., Lorenzi, J. C., . . . Bieniasz, P. D. (2020). Escape from neutralizing antibodies by SARS-CoV-2 spike protein variants. Elife, 9. doi:10.7554/eLife.61312
Whelan, S. P., Barr, J. N., & Wertz, G. W. (2004). Transcription and replication of nonsegmented negative-strand RNA viruses. Curr. Top. Microbiol. Immunol., 283:61-119., 61-119.
Wickersham, I. R., Finke, S., Conzelmann, K. K., & Callaway, E. M. (2007). Retrograde neuronal tracing with a deletion-mutant rabies virus. Nat Methods, 4(1), 47-49.
Wickersham, 1. R., Lyon, D. C., Barnard, R. J., Mori, T., Finke, S., Conzelmann, K. K., . . . Callaway, E. M. (2007). Monosynaptic restriction of transsynaptic tracing from single, genetically targeted neurons. Neuron, 53(5), 639-647.
Xie, X., Liu, Y., Liu, J., Zhang, X., Zou, J., Fontes-Garfias, C. R., . . . Shi, P.-Y. (2021). Neutralization of SARS-CoV-2 spike 69/70 deletion, E484K and N501Y variants by BNT162b2 vaccine-elicited sera. Nature Medicine. doi:10.1038/s41591-021-01270-4
Yinda, C. K., Port, J. R., Bushmaker, T., Offei Owusu, I., Purushotham, J. N., Avanzato, V. A., . . . Munster, V. J. (2021). K18-hACE2 mice develop respiratory disease resembling severe COVID-19. PLoS Pathog, 17(1), e1009195. doi:10.1371/journal.ppat.1009195
Zemp, F., Rajwani, J., & Mahoney, D. J. (2018). Rhabdoviruses as vaccine platforms for infectious disease and cancer. Biotechnol Genet Eng Rev, 34(1), 122-138. doi:10.1080/02648725.2018.1474320
Zhou, D., Dejnirattisai, W., Supasa, P., Liu, C., Mentzer, A. J., Ginn, H. M., . . . Screaton, G. R. (2021). Evidence of escape of SARS-CoV-2 variant B.1.351 from natural and vaccine-induced sera. Cell. doi:https://doi.org/10.1016/j.cell.2021.02.037

1. A composition comprising
(a) a recombinant rhabdovirus vector capable of forming a virus particle and expressing an immunogen of a betacoronavirus, wherein the immunogen comprises at the C-terminus a heterologous transmembrane anchor for the incorporation of the immunogen into (i) the cell membrane of infected cells, and (ii) the envelope of the virus particle, and/or
(b) a glycoprotein (G) protein gene deleted and in trans G protein complemented recombinant rhabdovirus vector capable of forming a virus-like particle (VLP) and expressing an immunogen of a betacoronavirus, wherein the immunogen comprises at the C-terminus a heterologous transmembrane anchor for the incorporation of the immunogen into (i) the cell membrane of infected cells, and (ii) the VLP.

2. The composition of claim 1, wherein the betacoronavirus is selected from SARS-CoV-2, MERS-CoV, SARS-CoV-1, OC43, and HKU1, and is preferably SARS-CoV-2.

3. The composition of claim 1, wherein the immunogen is the spike (S) protein or an immunogenic fragment thereof.

4. The composition of claim 3, wherein the immunogenic fragment consists of or comprises the spike receptor binding domain (RBD) and preferably-comprises a fragment as represented by SEQ ID NO: 1 or a variant of SEQ ID NO: 1 being with increasing preference at least 80%, at least 85%, at least 90% and at least 95% identical to SEQ ID NO: 1.

5. The composition of claim 1, wherein the rhabdovirus of (a) is preferably rhabdovirus vesicular stomatitis virus (VSV) or rabies virus (RABV), and/or the rhabdovirus of (b) is preferably a G protein gene deleted and pseudotyped VSV or RABV.

6. The composition of claim 1, wherein the immunogen of a betacoronavirus comprises at the N-terminus a signal peptide that promotes high-level translation into the endoplasmatic reticulum, wherein the signal peptide is preferably derived from an immunoglobulin, preferably from IgG and most preferably from the heavy chain of IgG.

7. The composition of claim 1, wherein the transmembrane anchor is derived from the stem of a rhabodovirus glycoprotein, and preferably derived from members of the Lyssavirus genus, more preferably derived from the rabies (RABV) vaccine strain SAD B19 (molecular clone SAD L16), and most preferably comprises or consists of the membrane proximal part of the G ectodomain, the trans-membrane and the cytoplasmic tail of the RABV SAD L16 G protein.

8. The composition of claim 1, wherein the recombinant rhabdovirus vector comprises at least two, preferably at least three copies of the nucleotide sequence encoding the immunogen of a betacoronavirus, wherein the vector comprises these copies at one site or at different sites of the vector.

9. The composition of claim 1, wherein the composition additionally comprises a VLP, wherein the VLP is preferably a G protein gene deleted rhabdovirus which is not complemented in trans with a functional G protein gene.

10. (canceled)

11. (canceled)

12. (canceled)

13. An immunogenic construct of a betacoronavirus, comprising
(i) the spike receptor binding domain (RBD) of a betacoronavirus, wherein the RBD preferably comprises SEQ ID NO: 1 or a variant of SEQ ID NO: 1 being with increasing preference at least 80%, at least 85%, at least 90% and at least 95% identical to SEQ ID NO: 1, and C-terminally thereof;
(ii) a transmembrane anchor being derived from the stem of a rhabodovirus glycoprotein, and preferably derived from members of the Lyssavirus genus, more preferably derived from the rabies (RABV) vaccine strain SAD B19 (molecular clone SAD L16), and most preferably comprising or consisting of the membrane proximal part of the G ectodomain, the trans-membrane and the cytoplasmic tail of the RABV SAD L16 G protein; and
(iii) at the N-terminus a signal peptide that promotes high-level translation into the endoplasmatic reticulum.

14. A nucleic acid molecule encoding an immunogenic construct of claim 13.

15. A virus vaccine vector or a plasmid or a DNA or RNA preparation expressing the immunogenic construct of claim 13 or comprising a nucleic acid molecule encoding the immunogenic construct.

16. A method for preventing or treating a betacoronavirus infection and a SARS-CoV-2 infection in a subject, comprising administering to the subject an effective amount of the composition of claim 1.

17. The method of claim 16, wherein the method further comprises a boost immunization with the composition or with virus-like particles presenting the chimeric immunogen in their envelope.

18. A method of detecting the presence of a betacoronavirus infection in a subject based on a sample, comprising using the composition of claim 1,
wherein the betacoronavirus infection is a SARS-CoV-2 infection, and
wherein the sample is a blood sample or a serum sample obtained from the subject.


SEQUENCE LISTING (27 sequences)

SEQ ID NO: 1 - protein, 228 aa, SARS-CoV2 isolate Wuhan-Hu-1
  RBD part of SARS-CoV2
QTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 2 - protein, 1273 aa, SARS-CoV2 isolate Wuhan-Hu-1
  Spike protein amino acid sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 3822 bp, SARS-CoV2 isolate Wuhan-Hu-1
  Spike protein gene nucleotide sequence
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 4 - DNA, 3822 bp, Artificial Sequence
  Human codon-optimized Spike gene nucleotide sequence
atgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctacagattcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacataa

SEQ ID NO: 5 - protein, 21 aa, Homo sapiens
  Signal peptide IgH HV 3-7 amino acid sequence
MELGLSWVFLVAILEGVQCEV

SEQ ID NO: 6 - DNA, 63 bp, Homo sapiens
  Signal peptide IgH HV 3-13 nucleotide sequence
atggaactgggactgtcttgggtgttcctggtggccattctggaaggcgtgcagtgcgaggtg

SEQ ID NO: 7 - protein, 21 aa, Homo sapiens
  Immunoglobulin heavy variable 3-21 (IGHV3-21)
MELGLRWVFLVAILEGVQCEV

SEQ ID NO: 8 - protein, 19 aa, Homo sapiens
  Immunoglobulin heavy variable 1-46 (IGHV1-46)
MDWTWRVFCLLAVAPGAHS

SEQ ID NO: 9 - protein, 15 aa, Homo sapiens
  High affinity immunoglobulin gamma Fc receptor I (FCGR1A)
MWFLTTLLLWVPVDG

SEQ ID NO: 10 - protein, 20 aa, Homo sapiens
  IgG receptor FcRn large subunit p51 (FCGRT)
MGVPRPQPWALGLLLFLLPG

SEQ ID NO: 11 - protein, 18 aa, Homo sapiens
  Serum albumin (ALB)
MKWVTFISLLFLFSSAYS

SEQ ID NO: 12 - protein, 28 aa, Homo sapiens
  Prolactin (PRL)
MNIKGSPWKGSLLLLLVSNLLLCQSVAP

SEQ ID NO: 13 - protein, 19 aa, Homo sapiens
  Azurocidin preproprotein (AZU1)
MTRLTVLALLAGLLASSRA

SEQ ID NO: 14 - protein, 118 aa, Artificial Sequence
  Stem/transmembrane anchor amino acid sequence
GSGSVIPLVHPLADPSTVFKDGDEAEDFVEVHLPDVHNQVSGVDLGLPNWGKYVLLSAGALTALMLIIFLMTCCRRVNRSEPTQHNLRGTGREVSVTPQSGKIISSWESHKSGGETRL

SEQ ID NO: 15 - DNA, 357 bp, Artificial Sequence
  Stem/transmembrane anchor nucleotide sequence
ggcagcggcagcgtgatccctctggttcatcctctggccgatcctagcaccgtgttcaaggatggggacgaagccgaggatttcgtggaagtgcatctgcccgatgtgcacaaccaggtgtcaggcgttgacctgggcctgcctaactggggcaaatacgtgctgctttctgccggcgctctgacagccctgatgctgatcatcttcctgatgacctgctgtcggagagtgaacagaagcgagcccactcagcacaacctgagaggcacaggcagagaagtgtccgtgacacctcagagcggcaagatcatcagcagctgggagagccacaagtctggcggcgagacaagactctga

SEQ ID NO: 16 - protein, 367 aa, Artificial Sequence
  Minispike amino acid sequence
MELGLSWVFLVAILEGVQCEVQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGSVIPLVHPLADPSTVFKDGDEAEDFVEVHLPDVHNQVSGVDLGLPNWGKYVLLSAGALTALMLIIFLMTCCRRVNRSEPTQHNLRGTGREVSVTPQSGKIISSWESHKSGGETRL

SEQ ID NO: 17 - DNA, 1104 bp, Artificial Sequence
  Minispike ORF nucleotide sequence
atggaactgggactgtcttgggtgttcctggtggccattctggaaggcgtgcagtgcgaggtgcagaccagcaactttagagtgcagcccaccgagagcatcgtgcggttccccaacatcaccaatctgtgccctttcggcgaggtgttcaacgccaccagattcgcctctgtgtacgcctggaaccggaagcggatcagcaattgcgtggccgactacagcgtgctgtacaacagcgccagcttcagcaccttcaagtgctacggcgtgtcccctaccaagctgaacgacctgtgcttcaccaacgtgtacgccgacagcttcgtgatcagaggcgacgaagtgcggcagattgcccctggacagacaggcaagatcgccgattacaactacaagctgcccgacgacttcaccggctgtgtgattgcctggaacagcaacaacctggacagcaaagtcggcggcaactacaactacctgtaccggctgttccggaagtccaacctgaagcctttcgagcgggacatcagcaccgagatctatcaggccggcagcaccccttgcaatggcgtggaaggcttcaactgctacttcccactgcagtcctacggcttccagcctacaaacggcgtgggctaccagccttacagagtggtggtgctgagcttcgagctgctgcatgctcctgccacagtgtgcggccctaagaaaagcaccaacctggtcaagaacaaatgcgtgaacttcggcagcggcagcgtgatccctctggttcatcctctggccgatcctagcaccgtgttcaaggatggggacgaagccgaggatttcgtggaagtgcatctgcccgatgtgcacaaccaggtgtcaggcgttgacctgggcctgcctaactggggcaaatacgtgctgctttctgccggcgctctgacagccctgatgctgatcatcttcctgatgacctgctgtcggagagtgaacagaagcgagcccactcagcacaacctgagaggcacaggcagagaagtgtccgtgacacctcagagcggcaagatcatcagcagctgggagagccacaagtctggcggcgagacaagactctga

SEQ ID NO: 18 - protein, 4 aa, Artificial Sequence
  Part of RBD construct
NITN

SEQ ID NO: 19 - protein, 6 aa, Artificial Sequence
  Part of RBD construct
HAPATV

SEQ ID NO: 20 - protein, 4 aa, Artificial Sequence
  Part of RBD construct
RVQP

SEQ ID NO: 21 - protein, 4 aa, Artificial Sequence
  Part of RBD construct
CVNF

SEQ ID NO: 22 - protein, 4 aa, Artificial Sequence
  Part of RBD construct
MFIF

SEQ ID NO: 23 - protein, 4 aa, Artificial Sequence
  Part of RBD construct
TSGS

SEQ ID NO: 24 - protein, 4 aa, Artificial Sequence
  Flanking residues
QTSN

SEQ ID NO: 25 - protein, 5 aa, Artificial Sequence
  Flanking residues
KCVNF

SEQ ID NO: 26 - protein, 4 aa, Artificial Sequence
  Short synthetic linker
GSGS

SEQ ID NO: 27 - protein, 15 aa, Artificial Sequence
  Two putative N-Glycosylation sites in the RBD part of the minispike
NITNLCPFGEVFNAT